(19) United States
(12) Patent Application Publication
(10) Pub. No.: US 2008/0293683 A1
(43) Pub. Date: Nov. 27, 2008

(54) Hormone Replacement Therapy

(75) Inventor: Bao Ting Zhu, Lexington, SC (US)

Correspondence Address: STINSON MORRISON HECKER LLP;ATTN: PATENT GROUP, 1201 WALNUT STREET, SUITE 2800, KANSAS CITY, MO, 64106-2150, US

(73) Assignee: University of Kansas Medical Center, Kansas City, KS (US)

(21) Appl. No.: 12/111,328

(22) Filed: Apr. 29, 2008

Related U.S. Application Data
(60) Provisional application No. 60/931,586, filed on May 24, 2007.

(51) Int. Cl. A61K 31/56; A61P 5/30

(52) U.S. Cl. 514/178; 514/169; 514/182

(57) ABSTRACT

A hormone replacement therapy formulation and method comprising selective estrogenic compounds which preferentially stimulate the estrogen receptor alpha over the estrogen receptor beta.

### CROSS-REFERENCE TO RELATED APPLICATIONS

This application is based on and claims priority to U.S. Provisional Application Ser. No. 60/931,586, filed on May 24, 2007 which is hereby incorporated herein by reference.

### STATEMENT REGARDING FEDERALLY SPONSORED RESEARCH OR DEVELOPMENT

The present invention was supported in part by the National Institutes of Health Grant No. RO1-CA-92391 and RO1-CA-97109, and the government may have certain rights in the invention.

### BACKGROUND OF THE INVENTION

Hormone replacement therapy has been known for some time. One particular aspect of hormone replacement therapy, known generally as estrogen replacement therapy, has been used for over 30 years for women during or following menopause. The reason for estrogen replacement, which is usually accomplished through transdermal absorption or orally, is to make up for the decline in, or the low level of, endogenous estrogens produced by the body. Typically, estrogen production decreases and then declines dramatically during and after menopause. It is during this time period that estrogen replacement is normally prescribed by a physician. However, estrogen replacement can be prescribed in other circumstances where other causes account for a decline in estrogen production or if estrogen is produced at a lower than desirable level. This could occur in women not yet in menopause.

The reasons for estrogen replacement, which have been substantiated by scientific research over a number of years, include the prevention and/or treatment of osteoporosis and cardiovascular disease, as well as preventing age-related decline in mental function. Estrogen replacement has also been used to decrease age-related changes in appearance.

For decades, the general scientific belief had been that “an estrogen is an estrogen,” i.e., all estrogens would exert similar pharmacological actions in the body. As such, the most commonly prescribed estrogen for estrogen replacement is actually concentrated from horse urine containing many estrogenic compounds sold under the name Premarin®. See generally, Hill et al., U.S. Pat. No. 6,855,703 entitled “Pharmaceutical compositions of conjugated estrogens and methods of analyzing mixtures containing estrogenic compounds.” Many physicians and others have objected to equine estrogen as being inappropriate for human use and even possibly dangerous because of the fact that many individual horse estrogens are not present in human bodies. There is also some evidence of the carcinogenic effect of equine estrogen.

In an attempt to duplicate or mimic the presence of natural estrogens in the human body by replacement therapy, some physicians in the 1980s began to prescribe combinations of the three classical human estrogens, namely, a combination of estrone (E₁), 17β-estradiol (E₂), and estriol (E₃). In addition, an estrogen formulation comprising 2-hydroxyestrone, 17β-estradiol, and estriol was proposed. See generally Wright, U.S. Pat. No. 6,911,438 entitled “Hormone replacement therapy.”

More recently, the role of estrogens in the body has been further elucidated. In particular, it has been found that many of the well-known hormonal actions of estrogens are mediated by specific estrogen receptors (“ERs”). The first high-affinity estrogen receptor, now commonly referred to as ERα, was cloned in 1986 from MCF-7 human breast cancer cells, which abundantly expressed this ER subtype. For nearly a decade after its cloning, it was believed that the estrogens signal through a single ER. However, a second ER (subtype β) was later identified in 1996 while studying the roles of estrogens in the prostate, gonads, and the immune system. The existence of two distinct ER subtypes indicated that the signaling pathways for endogenous estrogens are significantly more complex than previously thought.

The human ERα is a 66 kDa hormone-inducible transcription factor that can act positively or negatively in regulating the expression of genes involved in tissue growth and differentiation. The human ERβ is a 53 kDa hormone-inducible transcription factor that shares high degrees of sequence homology with the human ERα, especially in the DNA binding domain. Studies have shown that there are a number of functional similarities between human ERα and ERβ, and both receptor subtypes can bind 17β-estradiol (E₂) with similarly high affinities. See Kuiper et al., Comparison of the ligand binding specificity and transcript tissue distribution of estrogen receptors α and β, Endocrinology 138, 863-870 (1997); Katzenellenbogen et al., Hormone binding and transcription activation by estrogen receptors: analyses using mammalian and yeast systems, J. Steroid. Biochem. Mol. Biol. 47, 39-48 (1993). The activated ERα and ERβ (i.e., receptor bound with an agonist such as E₂) can form homodimers (ERα-ERα or ERβ-ERβ) or heterodimers (ERα-ERβ), and these dimerized ERs can bind to various estrogen response elements in highly similar fashions.

However, there are also significant differences noted for human ERα and ERβ. For example, it has been found that the tissue distribution pattern of these two ER subtypes is quite different. See Katzenellenbogen et al., A new actor in the estrogen receptor drama—Enter ER-β, Endocrinology 138, 861-862 (1997); Spong et al., Maternal estrogen receptor-β expression during mouse gestation, Am. J. Reprod. Immunol. 44, 249-252 (2000); Saunders et al., Expression of oestrogen receptor β (ERβ) in multiple rat tissues visualised by immunohistochemistry, J. Endocrinol. 154 R13-R16 (1997); Enmark et al., Human estrogen receptor β-gene structure, chromosomal localization, and expression pattern, J. Clin. Endocrinol. Metab. 82, 4258-4265 (1997); Shughrue et al., Comparative distribution of estrogen receptor-α and -βmRNA in the rat central nervous system, J. Comp. Neurol. 388, 507-525 (1997); Denger et al., Tissue-specific expression of human ERα and ERβ in the male, Mol. Cell Endocrinol. 178, 155-160 (2001). In addition, an earlier study has shown that 16β-hydroxyestradiol-17α (16β-OH-E₂-17α; commonly known as 16,17-epiestriol), an endogenous estrogen metabolite, has a preferential binding affinity for human ERβ over ERα. Hence, the possibility exists that some of the endogenously-formed estrogen metabolites/derivatives may have differential binding affinity for human ERα or ERβ, likely contributing to the differential activation of each signaling system in different target sites and/or under different physiological or pathophysiological conditions.

In recent years, the present inventor and others have made considerable effort to systematically characterize the complete profiles of the metabolites of E₂ and E₁ that are formed by human liver, non-hepatic tissues, as well as various recombinant human cytochrome P450 isoforms in vitro. A large number of endogenous estrogen metabolites have been identified. In the present invention, the ability of these metabolites to stimulate ERα and ERβ was investigated. Those results were then used to develop and select certain estrogenic compounds for use in estrogen replacement therapy that are believed to mimic the naturally occurring estrogens in non-pregnant pre-menopausal women.

### BRIEF SUMMARY OF THE INVENTION

The present invention is directed to a novel hormone replacement therapy formulation and method of using the hormone replacement therapy formulation to treat diseases, disorders, and conditions in women having low endogenous estrogen levels, especially in peri-menopausal and post-menopausal women.

In one aspect, the present invention is directed to a composition of matter comprising a mixture of estrogenic compounds endogenous to and normally circulating in the human female body.

In one aspect, the formulation consists essentially of one or more estrogenic compounds such that the relative binding affinity for ERα (“RBA_{α}”) of the estrogenic compounds compared to 17β-estradiol (E₂) is less than about 100%. For example, the estrogenic compounds may have an RBA_{α} which is less than about 30%, 25%, 20%, 15%, 10%, 5%, 4%, 2%, or 1% of 17β-estradiol (E₂). In a more preferred aspect, the formulation consists of estrogenic compounds such that the relative binding affinity for ERα (“RBA_{α}”) of the estrogenic compounds compared to 17β-estradiol (E₂) is less than about 100%.

In another aspect, the formulation consists essentially of one or more estrogenic compounds such that the relative binding affinity for ERβ (“RBA_{β}”) of the estrogenic compounds compared to 17β-estradiol (E₂) is less than about 100%. For example, the estrogenic compounds may have an RBA_{β} of less than about 10%, 5%, 4%, 3%, 2%, or 1% of 17β-estradiol (E₂). In a preferred aspect, the formulation consists of estrogenic compounds such that the relative binding affinity for ERβ (“RBA_{β}”) of the estrogenic compounds compared to 17β-estradiol (E₂) is less than about 100%.

In one aspect, the formulation consists essentially of one or more estrogenic compounds which preferentially stimulate the ERα over the ERβ. In another aspect, the formulation consists of estrogenic compounds which preferentially stimulate the ERα over the ERβ.

In a further aspect, the estrogenic compounds have a ratio of RBA_{α}/RBA_{β} which is greater than about 1, 2, 3, 4, 5, 6, 7, 8, 9, or 10. In another aspect, the ratio ranges between about 1:1 to 20:1, more preferably 2:1 to 10:1, with exemplary ranges being between about 1:1 to 3:1 and about 7:1 to 20:1.

In one aspect, the formulation comprises a mixture of at least one, two, or three estrogenic compounds that are endogenous to the non-pregnant pre-menopausal human female.

In another aspect, the formulation comprises a mixture of at least four or five estrogenic compounds that are endogenous to the non-pregnant pre-menopausal human female. Preferred estrogens that may be used in the present invention include estrone (RBA_{α}/RBA_{β} about 5), 1-methylestradiol (RBA_{α}/RBA_{β} about 1.8), 2-aminoestrone (RBA_{α}/RBA_{β} about 7.5), 2-nitroestrone (RBA_{α}/RBA_{β} about 3), 2-hydroxyestrone (RBA_{α}/RBA_{β} about 10), 2-methoxyestradiol (RBA_{α}/RBA_{β} about 2), 2-bromoestradiol (RBA_{α}/RBA_{β} about 10), 4-nitroestrone (RBA_{α}/RBA_{β} about 10); 4-hydroxyestrone (RBA_{α}/RBA_{β} about 2), 4-hydroxyestradiol (RBA_{α}/RBA_{β} about 1.3), 4-methoxyestradiol (RBA_{α}/RBA_{β} about 2), 6-ketoestrone ((RBA_{α}/RBA_{β} about 2), 6α-hydroxyestradiol (RBA_{α}/RBA_{β} about 1.5), 6-ketoestradiol (RBA_{α}/RBA_{β} about 1.3), 6-ketoestriol (RBA_{α}/RBA_{β} about 8.3), 6-ketoestradiol-17α (RBA_{α}/RBA_{β} about 2), 7-dehydroestradiol (RBA_{α}/RBA_{β} about 1.3), 7-dehydroestradiol-17α (RBA_{α}/RBA_{β} about 1.3), 2-hydroxyestriol (RBA_{α}/RBA_{β} about 2), 17)-estradiol 11-acetate (RBA_{α}/RBA_{β} of 1.2), 11β-methoxyethynyl estradiol (RBA_{α}/RBA_{β} of about 1.8), estetrol (RBA_{α}/RBA_{β} of about 1.3), and 16β-hydroxyestradiol (RBA_{α}/RBA_{β} of about 1.3), 17α-estradiol (RBA_{α}/RBA_{β} about 7.3), 17α-ethynylestradiol (RBA_{α}/RBA_{β} about 3.6).

The estrogenic compounds may also take the form of prodrugs. For example, 2-methoxyestrone is readily converted in the body to 2-methoxyestradiol (RBA_{α}/RBA_{β} about 2) by the enzyme 17β-hydroxysteroid dehydrogenase. As another example, estrone-3-sulfate, which has virtually no estrogen receptor binding affinity, can be readily hydrolyzed into estrone (RBA_{α}/RBA_{β} about 5). Still as another example, 17α-estradiol-3-sulfate or 17α-estradiol-17-sulfate is rapidly converted to 17α-estradiol (17α-E₂).

Most preferred estrogenic compounds are selected from the group consisting of comprises estrone (E₁), 17α-estradiol (17α-E₂), 2-hydroxyestrone (2-OH-E₁), 2-methoxyestrone (2-MeO-E₁), and/or 2-methoxyestradiol (2-MeO-E₂), as well as their sulfated or glucuronidated conjugates. In another aspect, the formulation consists essentially of estrone (E₁), 17α-estradiol (17α-E₂), 2-hydroxyestrone (2-OH-E₁), 2-methoxyestrone (2-MeO-E₁), and/or 2-methoxyestradiol (2-MeO-E₂), as well as their sulfated or glucuronidated conjugates.

The estrogenic compounds in the mixture may be present in a chemically pure form, or as prodrugs, especially sulfated or glucuronidated conjugates, and their pharmaceutically acceptable salts. Thus, for example, the formulation may include pharmaceutically acceptable salts of conjugated estrone (E₁), conjugated 17α-estradiol (17α-E₂), conjugated 2-hydroxyestrone (2-OH-E₁), conjugated 2-methoxyestrone (2-MeO-E₁), and/or conjugated 2-methoxy-estradiol (2-MeO-E₂). In one aspect, the pharmaceutically acceptable salt is a sodium salt.

According to embodiments of the present invention, the formulation may include the pharmaceutically acceptable salts of estrone sulfate, 17α-estradiol sulfate, 2-hydroxyestrone sulfate, 2-methoxyestrone sulfate, and/or 2-methoxy-estradiol sulfate.

According to still other embodiments of the present invention, the formulation may include the sodium salts of estrone sulfate, 17α-estradiol sulfate, 2-hydroxyestrone sulfate, 2-methoxyestrone sulfate, and/or 2-methoxy-estradiol sulfate.

In another aspect, the invention provides a method of treating subjects in need of treatments of various diseases and disorders associated with low levels of estrogenic compounds. The method comprises administering an effective amount the estrogenic compounds of the present invention (and formulations containing the same) to a subject in need thereof. Examples of treatments that are addressed by the compositions of the invention include vasomotor symptoms, atrophic vaginitis, and osteoporosis.

In another aspect, the estrogenic compounds of the present invention are co-administered with one or more protesting, such as progesterone.

Additional aspects of the invention, together with the advantages and novel features appurtenant thereto, will be set forth in part in the description which follows, and in part will become apparent to those skilled in the art upon examination of the following, or may be learned from the practice of the invention. The objects and advantages of the invention may be realized and attained by means of the instrumentalities and combinations particularly pointed out in the appended claims.

### BRIEF DESCRIPTION OF THE DRAWINGS

FIG. 1 shows the competition of [³H]E₂ binding to recombinant human ERα and ERβ by E₂, E₁, E₃ (estriol or 16α-OH-E₂), or E₂-17α. The conditions for the in vitro ER binding assay were described in detail herein. For the data shown in panels A-D, the concentration of the radioactive ligand [³H]E₂ was 10 nM, and the concentrations of the competing estrogens were 0, 0.24, 0.98, 3.9, 15.6, 62.5, 250, and 100 nM. For the data shown in panels E and F, the concentrations of the radioactive ligand [³H]E₂ were 0.025, 0.1, 0.2, 0.4, 0.8, 1.6, 3.2, 6.25, 12.5, and 25 nM. The non-specific binding was determined in the presence of 400-fold excess of cold E₂. The K_{D} values for ERα and ERβ were calculated according the S-shaped binding curves (curve regression analysis). Abbreviations used: (“TB”), total binding; (“NSB”), non-specific binding; (“SB”), specific binding. Each data point in panels A-F was the mean of duplicate measurements.

FIG. 2 shows competition of the binding of [³H]E₂ to human ERα and ERβ by various catechol estrogens and methoxyestrogens. The conditions for the in vitro ER binding assay were described in detail herein. The concentration of the radioactive ligand [³H]E₂ was 10 nM, and the concentrations of the competing estrogens were 0, 0.24, 0.98, 3.9, 15.6, 62.5, 250, and 100 nM. Each data point was the mean of duplicate measurements.

FIG. 3 shows the competition of the binding of [³H]E₂ to human ERα and ERβ by several other A-ring analogs (most of them are synthetic analogs). The conditions for the receptor binding assay were the same as described in the legend to FIG. 2.

FIG. 4 shows the competition of the binding of [³H]E₂ to human ERα and ERβ by several B-ring and C-ring substitution metabolites or derivatives. The conditions for the receptor binding assay were the same as described in the legend to FIG. 2.

FIG. 5 shows the competition of the binding of [³H]E₂ to human ERα and ERβ by several B-ring and C-ring dehydroestrogen metabolites or derivatives. The conditions for the receptor binding assay were the same as described in the legend to FIG. 2.

FIG. 6 shows the competition of the binding of [³H]E₂ to human ERα and ERβ by several D-ring metabolites or derivatives. The conditions for the receptor binding assay were the same as described in the legend to FIG. 2. Note that for 17-desoxy-E₂ and 1,3,5(10),16-estratetraen-3-ol, two more lower concentrations (0.015 and 0.06 nM) were also assayed.

FIG. 7 shows the competition of the binding of [³H]E₂ to human ERα and ERβ by several antiestrogens, phytoestrogens, and stilbene estrogens. The conditions for the receptor binding assay were the same as described in the legend to FIG. 2.

### DETAILED DESCRIPTION OF PREFERRED EMBODIMENT

The present invention is directed to a novel hormone replacement therapy formulation, and method of using the hormone replacement therapy formulation, to treat diseases, disorders, and conditions associated with low estrogen levels in non-pregnant women, especially post-menopausal and peri-menopausal women.

In general, the symptoms associated with treatment methods of the present invention include, but are not limited to osteoporosis, coronary heart disease, breast tenderness, oedema, fatigue, hot flashes, sweating, headache, shortness of breath, depression, night sweats, anxiety, sleep disorders, vaginal dryness, vaginal shrinkage, dry skin and hair, hair loss, mood swings, urinary incontinence, nausea, heart palpitations, short-term memory loss, frequent urinary tract infections, yeast infections, painful intercourse, decreased sexual activity, and inability to reach orgasm.

In a preferred aspect, hormone replacement therapy formulation consists essentially of estrogenic compounds such that: (1) the relative binding affinity for ERα (“RBA_{α}”) of the estrogenic compounds compared to 17β-estradiol (E₂) is less than about 100%; (2) the relative binding affinity for ERβ (“RBA_{β}”) of the estrogenic compounds compared to 17β-estradiol (E₂) is less than about 100%; and/or (3) the estrogenic compounds preferentially stimulate the ERα over the ERβ such that the ratio of RBA_{α}/RBA_{β} is greater than about 1.

In one aspect, the formulation comprises a mixture of at least three estrogenic compounds and/or pharmaceutically acceptable conjugates. In another aspect, the formulation comprises a mixture of at least five estrogenic compounds and/or their pharmaceutically acceptable conjugates. Especially preferred estrogenic compounds are estrone (E₁), 17α-estradiol (17α-E₂), 2-hydroxyestrone (2-OH-E₁), 2-methoxyestrone (2-MeO-E₁), and 2-methoxyestradiol (2-MeO-E₂), and pharmaceutically acceptable salts or prodrugs thereof, including their sulfated or glucuronidated conjugates. The structures of these preferred compounds are below:

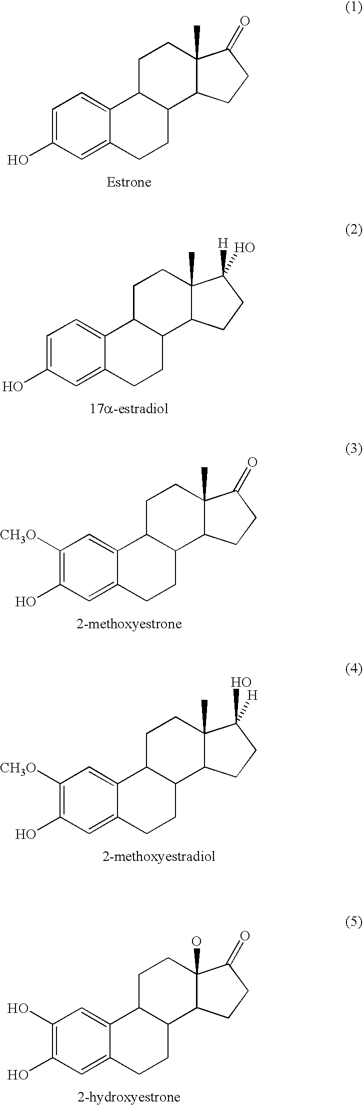

The estrogenic compounds are preferably in the form of conjugated estrogens, which function as prodrugs. Other pharmaceutically acceptable prodrugs may also be used. The conjugates may be any suitable conjugate known by those skilled in the art, including, but not limited to, glucuronide and sulfate. The estrogenic compounds may also be present as pharmaceutically acceptable salts of the conjugated estrogens. The pharmaceutically acceptable salts may be various salts understood by those skilled in the art, including, but not limited to, sodium salts, calcium salts, magnesium salts, lithium salts, and amine salts such as piperazine salts. The most preferred salts are sodium salts.

The estrogenic compounds are administered in a therapeutically effective amount to treat the specified condition, for example in a daily dose preferably ranging from about 1 to about 1000 mg per day, and more preferably about 5 to about 200 mg per day, given in a single dose or 2-4 divided doses. The plasma concentration for each of the estrogenic compounds preferably ranges between about 10 and 50 pg/ml. The exact dose, however, is determined by the attending clinician and is dependent on such factors as the potency of the compound administered, the age, weight, condition, and response of the patient.

The term “co-administered” means the administration of the selected estrogenic compounds (or other agents, such as progestins) to a subject by combination in the same pharmaceutical composition or separate pharmaceutical compositions. Thus, co-administration involves administration at the same time of a single pharmaceutical composition comprising the estrogenic compounds or administration of two or more different compositions to the same subject at the same or different times.

The terms “comprising” or “having” indicate that any estrogenic compounds or steps can be present in addition to those recited in the hormone replacement therapy formulations and methods.

The term “consists essentially of” or “consisting essentially of” indicates that unlisted ingredients or steps that do not materially affect the basic and novel properties of the invention can be employed in addition to the specifically recited estrogenic compounds. Typically, this means that the hormone replacement therapy does not contain estrogenic compounds in an amount that preferentially stimulates the ERβ over the ERα more than those in the normal pre-menopausal non-pregnant human female. In a preferred aspect, the hormone replacement therapy compositions do not contain any estrogenic compounds that preferentially stimulate the ERβ over the ERα.

The term “consists of” or “consisting of” indicates that only the recited estrogenic compounds or steps are present, but does not foreclose the possibility that equivalents of the ingredients or steps can substitute for those specifically recited.

The term “menopause” is used throughout the specification to describe the period in a woman's life between the ages of approximately 45 and 50 (but not always) after which menstruation (menses) naturally ceases. The symptomology associated with menopause which is particularly relevant to the present invention includes bone loss associated with osteoporosis, for example.

The terms peri-menopausal refers to that time in a women's life between pre-menopause (the reproductive years) and post-menopause. This time period is usually between the ages of 40-60, but more often several years on either side of 45 to 50 years of age. This period is characterized by a rapid change in the hormonal balance in a woman. The hallmark of the ending of the peri-menopausal period and the beginning of the post-menopausal period is the cessation of ovarian function or its inability to regulate the previously normal ovulation cycle in the woman. This cessation of function is clinically marked by the cessation of menses of a period of one year or more. The time period over which this cessation of ovarian function persists, i.e., the peri-menopausal time, is usually not a sudden or rapid event. The peri-menopausal state can last from a few months to more typically a year or more.

The term “patient” is used throughout the specification to describe an animal, preferably a human, to whom treatment, including prophylactic treatment, with the estrogenic compound formulation according to the present invention is provided. For treatment of the symptomology, conditions, or disease states which are specific for a specific animal such as a human patient, the term patient refers to that specific animal. In most instances in the present invention, the patient is a human female exhibiting symptomology associated with menopause. While patients of the present invention are preferably post-menopausal woman, it will be appreciated that estrogen replacement can be prescribed in other circumstances where other causes account for a decline in estrogen production or if estrogen is produced at a lower than desirable level. This could occur in women not yet in menopause.

The term “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “prodrug” means a covalently-bonded derivative or carrier of the parent estrogenic compound which undergoes at least some biotransformation prior to exhibiting its pharmacological effect(s). In general, such prodrugs have metabolically cleavable groups and are rapidly transformed in vivo to yield the parent compound, for example, by hydrolysis in blood. The prodrug is formulated with the objectives of improved chemical stability, improved patient acceptance and compliance, improved bioavailability, prolonged duration of action, improved organ selectivity, improved formulation (e.g., increased hydrosolubility), and/or decreased side effects (e.g., toxicity). In general, prodrugs themselves have weak or no biological activity and are stable under ordinary conditions. Prodrugs can be readily prepared from the parent compounds using methods known in the art, such as those described in A Textbook of Drug Design and Development, Krogsgaard-Larsen and H. Bundgaard (eds.), Gordon & Breach, 1991, particularly Chapter 5: “Design and Applications of Prodrugs”; Design of Prodrugs, H. Bundgaard (ed.), Elsevier, 1985; Prodrugs: Topical and Ocular Drug Delivery, K. B. Sloan (ed.), Marcel Dekker, 1998; Methods in Enzymology, K. Widder et al., (eds.), Vol. 42, Academic Press, 1985, particularly pp. 309-396; Burger's Medicinal Chemistry and Drug Discovery, 5th Ed., M. Wolff (ed.), John Wiley & Sons, 1995, particularly Vol. 1 and pp. 172-178 and pp. 949-982; Pro-Drugs as Novel Delivery Systems, T. Higuchi and V. Stella (eds.), Am. Chem. Soc., 1975; and Bioreversible Carriers in Drug Design, E. B. Roche (ed.), Elsevier, 1987, each of which is incorporated herein by reference in their entireties.

The term “therapeutically effective amount” is understood to mean a sufficient amount of an estrogenic compound(s) or composition that will positively modify the symptoms and/or condition to be treated. The therapeutically effective amount can be readily determined by those of ordinary skill in the art, but of course will depend upon several factors. For example, one should consider the condition and severity of the condition being treated, the age, body weight, general health, sex, diet, and physical condition of the patient being treated, the duration of the treatment, the nature of concurrent therapy, the particular active ingredient being employed, the particular pharmaceutically-acceptable excipients utilized, the time of administration, method of administration, rate of excretion, drug combination, and any other relevant factors.

The estrogenic compounds are preferably administered to the patient in a continuous uninterrupted fashion. In one aspect, the frequency of administration is at least once daily. The term “continuous” as applied in the specification means that the dosage is administered at least once daily. The term “uninterrupted” means that there is no break in the treatment, and that the treatment is administered at least once daily in perpetuity until the entire treatment is ended.

Techniques for preparing the formulations comprising the estrogenic compounds of the present invention are set forth, for example, in Huber et al., U.S. Pat. No. 5,908,638 entitled “Pharmaceutical compositions of conjugated estrogens and methods for their use”; Potter et al., U.S. Pat. No. 6,326,366 entitled “Hormone Replacement Therapy”; Hochberg, U.S. Pat. No. 6,476,012 entitled “Estradiol-16α-Carboxylic Acid Esters as Locally Active Estrogens”; Luo et al., U.S. Pat. No. 6,562,370 entitled “Transdermal Administration of Steroid Drugs Using Hydroxide-Releasing Agents as Permeation Enhancers”; Casper et al., U.S. Pat. No. 6,747,019 entitled “Low Dose Estrogen Interrupted Hormone Replacement Therapy”; Lanquetin et al., U.S. Pat. No. 6,831,073 entitled “Hormonal Composition Consisting of an Estrogen Compound and of a Progestational Compound”; Hill et al., U.S. Pat. No. 6,992,075 entitled “C(14) Estrogenic Compounds” which are incorporated by reference. The hormone replacement therapy compositions may further comprise one or more pharmaceutically acceptable carriers, one or more excipients, and/or one or more additives. The hormone replacement therapy compositions may comprise about 1 to about 99 weight percent of the estrogenic compounds.

Useful pharmaceutically acceptable carriers can be solid, liquid, or gas. Non-limiting examples of pharmaceutically acceptable carriers include solids and/or liquids such as magnesium carbonate, magnesium stearate, talc, sugar, lactose, ethanol, glycerol, water, and the like. The amount of carrier in the formulation can range from about 5 to about 99 weight percent of the total weight of the treatment composition or therapeutic combination. Non-limiting examples of suitable pharmaceutically acceptable excipients and additives include non-toxic compatible fillers, binders such as starch, polyvinyl pyrrolidone or cellulose ethers, disintegrants such as sodium starch glycolate, crosslinked polyvinyl pyrrolidone or croscarmellose sodium, buffers, preservatives, anti-oxidants, lubricants, flavorings, thickeners, coloring agents, wetting agents such as sodium lauryl sulfate, emulsifiers, and the like. The amount of excipient or additive can range from about 0.1 to about 95 weight percent of the total weight of the treatment composition or therapeutic combination. One skilled in the art would understand that the amount of carrier(s), excipients, and additives (if present) can vary. Further examples of pharmaceutically acceptable carriers and methods of manufacture for various compositions can be found in A. Gennaro (ed.), Remington: The Science and Practice of Pharmacy, 20th Edition, (2000), Lippincott Williams & Wilkins, Baltimore, Md., which is periodically updated.

Useful solid form preparations include powders, tablets, dispersible granules, capsules, cachets, and suppositories. Useful liquid form preparations include solutions, suspensions, and emulsions. As an example may be mentioned water or water-propylene glycol solutions for parenteral injection or addition of sweeteners and opacifiers for oral solutions, suspensions, and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g. nitrogen.

Also useful are solid form preparations which are intended to be converted, shortly before use, to liquid form preparations for either oral or parenteral administration. Such liquid forms include solutions, suspensions, and emulsions.

The compounds of the invention may also be deliverable transdermally. The transdermal compositions can take the form of creams, lotions, aerosols, and/or emulsions can be included in a transdermal patch of the matrix or reservoir type as are conventional in the art for this purpose. Preferably, the compound is administered orally.

In one aspect, the delivery vehicle or formulation of the invention preferably provides for administration of estrogenic composition by an oral, subcutaneous, intravenous, intramuscular, intraperitoneal, intrabuccal, vaginal, or transdermal route. Preferably, the carrier vehicle or device for each component is selected from a wide variety of materials and devices which are already known per se or may hereafter be developed which provide for controlled release of the compositions in the particular physiological environment. In particular, the carrier vehicle of the delivery system is selected such that near zero-order release of the components of the regimen is achieved. In the context of the present invention, the carrier vehicle should therefore also be construed to embrace particular formulations of the compositions which are themselves suitable for providing near zero-order release. A targeted steady-state release can be obtained by suitable adjustment of the design or composition of the delivery system. Known devices suitable for use as a delivery system in accordance with the present invention include, for example, drug-delivery pump devices providing near zero-order release of the components of the regimen.

The following examples are provided to illustrate the present invention and are not intended to limit the scope thereof.

### Example 1

### Determination of Estrogen Receptor Activation

This example is set forth in Zhu et al., Quantitative Structure-Activity Relationship of Various Endogenous Estrogen Metabolites for Human Estrogen Receptor α and β Subtypes: Insights into the Structural Determinants Favoring a Differential Subtype Binding, Endocrinology 147, 4132-4150 (2006), which is incorporated by reference.

In this example, endogenous E₁ and E₂ metabolites, along with some of their synthetic analogs and phytoestrogens (structures shown in below in Table 1), were compared for their binding affinities for human ERα and ERβ. The recombinant human ERs used in the present study were produced in a baculovirus expression system that yielded soluble, functionally-active recombinant ER proteins with post-translational modification patterns (mainly phosphorylations and acetylations) similar to those found in mammalian cells. See Reid et al., Human estrogen receptor-α: Regulation by synthesis, modification and degradation, Cell. Mol. Life. Sci. 59, 821-831 (2002).

TABLE 1
Structure of E₂ and Various Natural or Synthetic Estrogens

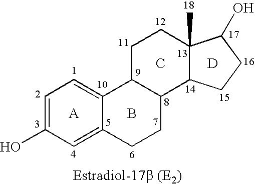

| | |
|---|---|
| 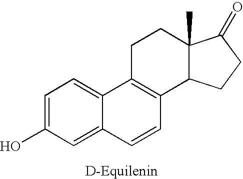 | 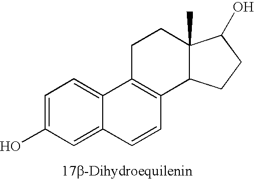 |
| 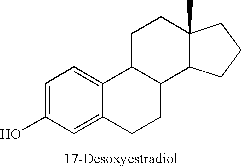 | 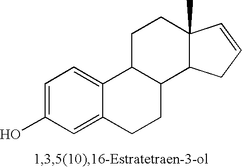 |
| 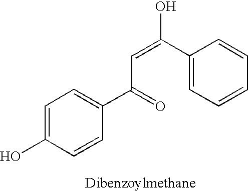 | 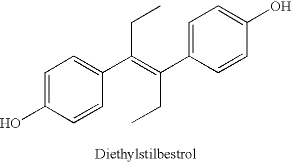 |
| 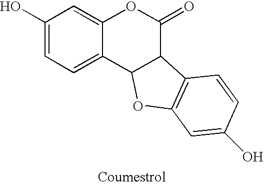 | 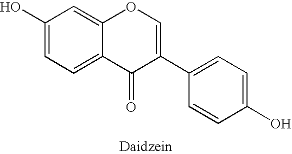 |

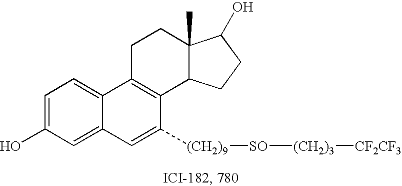

| | |
|---|---|
| 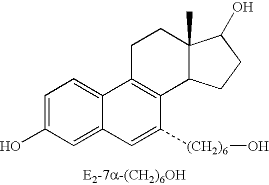 | 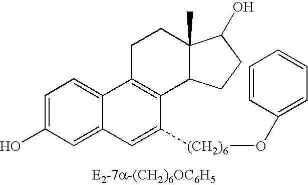 |
| 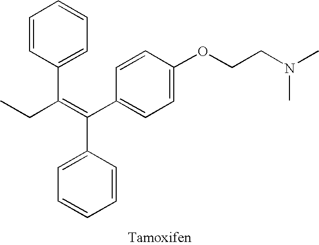 | 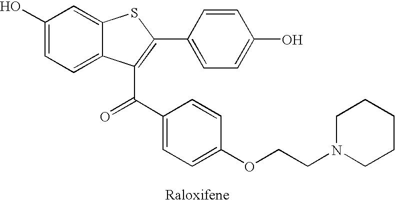 |
| 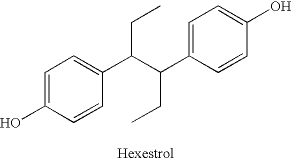 | 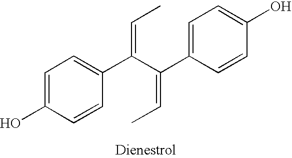 |
| 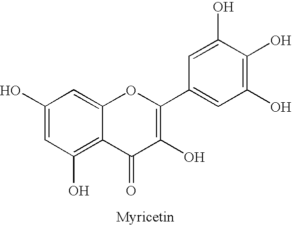 | 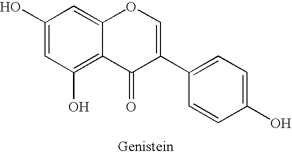 |

Chemicals and Reagents

E₂, E₁, and most of their metabolites and derivatives listed in Table 2 were obtained from Steraloids (Newport, R.I.). 7α-(6-Hydroxyhexanyl)-17β-estradiol [E₂-7α-(CH₂)₆OH] and 7α-(6-benzyloxyhexanyl)-17β-estradiol [E₂-7α-(CH₂)₆OC₆H₅] were chemically zed according to Jiang et al., Synthesis of 7α-substituted derivatives of 17β-estradiol, Steroids 71 334-342 (2006). Dithiothreitol, glycerol, and Tris-HCl were obtained from the Sigma Chemical Co. (St. Louis, Mo.). Hydroxylapatite and bovine serum albumin (BSA) were obtained from Calbiochem (through EMD Biosciences, Inc. San Diego, Calif.). [2,4,6,7,16,17-³H]E₂ (specific activity of 115 Ci/mmol) was obtained from NEN Life Sciences (Boston, Mass.), and it was purified in our laboratory using a high-pressure liquid chromatography (HPLC)-based method prior to its use in the in vitro receptor binding assays. See Lee et al. Characterization of the oxidative metabolites of 17β-estradiol and estrone formed by 15 selectively expressed human cytochrome p450 isoforms, Endocrinology 144, 3382-3398 (2003).

The recombinant human ERα and ERβ proteins and bovine serum albumin (BSA) were obtained from PanVera Corporation (Madison, Wis.). According to the supplier, the recombinant human ERα and ERβ were produced in a baculovirus-mediated expression system, and they were soluble and functionally active, with post-translational modifications similar to those found in mammalian cells. See Reid et al., Human estrogen receptor-α: Regulation by synthesis, modification and degradation, Cell. Mol. Life. Sci. 59, 821-831 (2002).

ERα and ERβ Binding Assays

The following buffer solutions were used in the ER binding assays, and they were prepared beforehand and stored at 4° C. The binding buffer consisted of 10% glycerol, 2 mM dithiothreitol, 1 mg/mL BSA and 10 mM Tris-HCl at pH 7.5. The ERα washing buffer contained 40 mM Tris-HCl and 100 mM KCl (pH 7.4), but the ERG washing buffer contained only 40 mM Tris-HCl (adjusted to pH 7.4). The 50% hydroxylapatite slurry was prepared first by vigorously mixing 10 g hydroxylapatite with 60 mL of the Tris-HCl solution (50 mM, pH 7.4). Hydroxylapatite was then allowed to settle for 20 minutes at room temperature, and the supernatant was decanted. The above procedures were repeated 10 times, and afterwards hydroxylapatite was kept in the 50 mM Tris-HCl solution overnight at 4° C. Hydroxylapatite slurry was then adjusted to an approximate final concentration of 50% (v/v) using the same Tris-HCl solution and stored at 4° C., and the slurry was stable for up to several months.

On the day of performing the ER binding assay, [³H]E₂ solution was freshly diluted in the binding buffer, and an aliquot (45 μL) of the [³H]E₂ solution was added to a 1.5 mL microcentrifuge tube, giving a final [³H]E₂ concentration at 10 nM. Each of the competing ligands (in 50 μL volume) was then added to the mixture for the intended final concentrations at 0, 0.24, 0.98, 3.9, 15.6, 62.5, 250, and 1000 nM. Note that all of the estrogens were initially dissolved in pure ethanol to a stock concentration of 1 mM, and then further diluted to 100 μM with 20% aqueous ethanol. In this way, the final ethanol concentration in the incubation mixture was less than 0.2%. Immediately before the addition of the ERα or ERβ protein, it was diluted in the binding buffer and mixed gently with repetitive pipettings. An aliquot (5 μL) of the diluted ERα or ERβ solution was precisely added to the mixture containing 45 μL of the [³H]E₂ and 50 μL of the competing ligand, giving a final receptor concentration of 1-2 fmol/mL. The incubation mixture was then mixed gently and thoroughly with repetitive pipettings. Nonspecific binding (NSB) by the [³H]E₂ was determined in separate tubes by inclusion of a 400-fold concentration of the nonradioactive E₂ (at a final concentration of 4 μM). Based on UV spectrometric monitoring of E₂ in water, this estrogen at 4 μM concentration (the highest steroid concentration used) appeared to be readily soluble in the aqueous solution. The binding mixture was incubated at room temperature for two hours. At the end of the incubation, 100 μL of the hydroxylapatite slurry was added to each tube and the tubes were incubated on ice for 15 minutes with 3 times of brief vortexing. An aliquot (1 mL) of the appropriate washing buffer was added, mixed, and centrifuged at 10,000 g for five minutes, and the supernatants were discarded. This wash step was repeated twice. Hydroxylapatite pellets were then resuspended in 200 μL ethanol (followed by another rinse with 200 μL ethanol), and then the content was transferred to scintillation vials (containing 4 mL of the scintillation fluid) for measurement of ³H-radioactivity with a liquid scintillation counter (Packard Tri-CARB 2900 TR; Downers Grove, Ill.).

To calculate the specific binding (pmol/mL) of the human ERα or ERβ protein at each concentration point, the following equation was used:

$$ER α or ER β = \frac{( d . p . m . for total binding - d . p . m . for NSB ) \times dilution factor}{final volume of the mixture \times ( d . p . m . / pmol [ {}^{3} H ] {E}_{2} )}$$

The IC₅₀ value for each competing estrogen was calculated according to the sigmoidal inhibition curve, and the relative binding affinity (RBA) was calculated against E₂ using the following equation:

$$RBA = \frac{{IC}_{50} for {E}_{2}}{{IC}_{50} for the test compound}$$

It should be noted that the absolute IC₅₀ values are affected by the concentrations of the radioligand ([³H]E₂) used. When a lower radioligand concentration is used, the corresponding IC₅₀ value will also be relatively lower, but when the radioligand concentration increases, the corresponding IC₅₀ value will also increase. Because the radioligand [³H]E₂ concentration used in this study was 10 nM (which was 10-100 times higher than the previously-reported K_{D} values for human ERα), the absolute IC₅₀ values would also be higher if they were compared with values reported in some of the earlier studies when lower concentrations of the radioligand were used. The reason that a higher concentration of [³H]E₂ was used was simply because it would yield more reproducible readings of the radioactivity counts. Since the RBA value is a parameter that is independent of the radioligand concentration used, we thus have placed more emphasis on the RBA values instead of the absolute IC₅₀ values in interpreting the physiological meanings of the data from in vitro receptor competition assays.

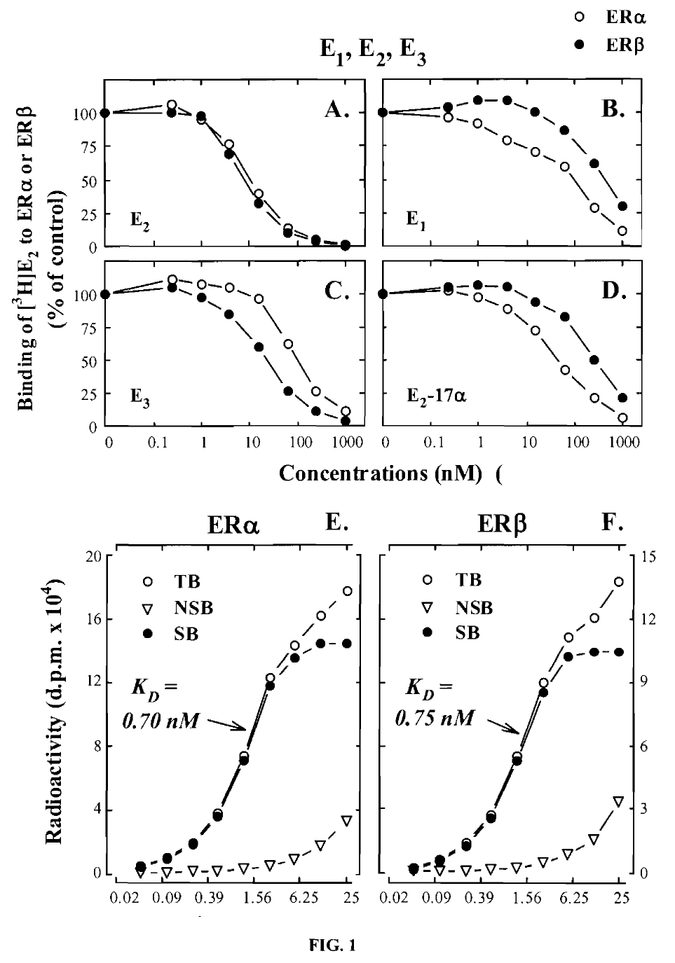

TABLE 2
The IC₅₀ and RBA values of various hydroxylated, keto, and
dehydrogenated metabolites of E₂ and E₁ as well as some other natural
or synthetic derivatives for the recombinant human ER₂ and ER₁.

| | | | ERα | | ERβ | | |
|---|---|---|---|---|---|---|---|
| | | Figure | IC₅₀ | | IC₅₀ | RBA_{β} | RBA_{α}/ |
| Chemical names | Abbreviations | No. | (nM) | RBA_{α} % | (nM) | (%) | RBA_{β} |
| Estradiol-17β (or Estradiol) | E₂ | FIG. 1A | 11.2 | 100 | 8.9 | 100 | 1 |
| Estrone | E₁ | FIG. 1B | 112.2 | 10 | 446.7 | 12 | 5 |

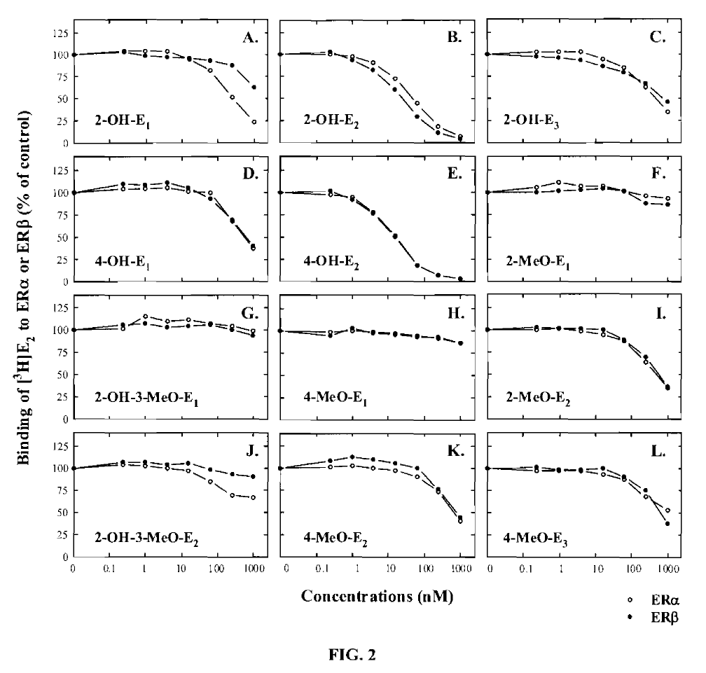

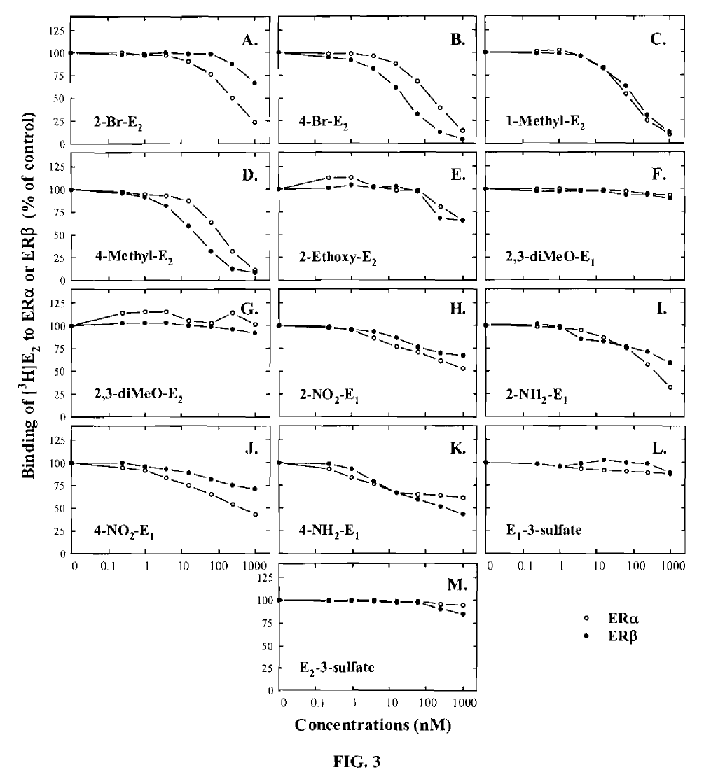

A-RING METABOLITES

| | | | | | | | |
|---|---|---|---|---|---|---|---|
| 1-Methylestradiol | 1-Methyl-E₂ | FIG. 3C | 79.4 | 14 | 112.2 | 8 | 1.8 |
| 2-Aminoestrone | 2-NH₂-E₁ | FIG. 3I | 398.1 | 3 | 2511.9 | 0.4 | 7.5 |
| 2-Nitroestrone | 2-NO₂-E₁ | FIG. 3H | 1584.9 | 1 | ND | ND | ND |
| 2-Hydroxyestrone | 2-OH-E₁ | FIG. 2A | 316.2 | 4 | 1995.3 | 0.4 | 10 |
| 2-Hydroxyestradiol | 2-OH-E₂ | FIG. 2B | 50.1 | 22 | 25.1 | 35 | 0.6 |
| 2-Hydroxyestriol | 2-OH-E₃ | FIG. 2C | 501.2 | 2 | 794.3 | 1 | 2 |
| 2-Methoxyestrone | 2-MeO-E₁ | FIG. 2F | ND | ND | ND | ND | ND |
| 2-Methoxyestradiol | 2-MeO-E₂ | FIG. 2I | 501.2 | 2 | 631.1 | 1 | 2 |
| 2-Ethoxyestradiol | 2-Ethoxy-E₂ | FIG. 3E | ND | ND | ND | ND | ND |
| Estrone 2,3-dimethyl ether | 2,3-diMeO-E₁ | FIG. 3F | ND | ND | ND | ND | ND |
| 17β-Estradiol 2,3-dimethyl ether | 2,3-diMeO-E₂ | FIG. 3G | ND | ND | ND | ND | ND |
| 2-Hydroxyestrone 3-methyl ether | 2-OH-3-MeO-E₁ | FIG. 2G | ND | ND | ND | ND | ND |
| 2-Hydroxyestradiol 3-methyl ether | 2-OH-3-MeO-E₂ | FIG. 2I | ND | ND | ND | ND | ND |
| 2-Bromoestradiol | 2-Br-E₂ | FIG. 3A | 281.1 | 4 | 2511.1 | 0.4 | 10 |
| 4-Aminoestrone | 4-NH₂-E₁ | FIG. 3K | 354.8 | 3 | ND | ND | ND |
| 4-Nitroestrone | 4-NO₂-E₁ | FIG. 3J | 446.7 | 3 | ND | ND | ND |
| 4-Hydroxyestrone | 4-OH-E₁ | FIG. 2D | 708.1 | 2 | 708.1 | 1 | 2 |
| 4-Hydroxyestradiol | 4-OH-E₂ | FIG. 2E | 15.9 | 70 | 15.9 | 56 | 1.3 |
| 4-Methylestradiol | 4-Methyl-E₂ | FIG. 3D | 125.9 | 9 | 25.1 | 35 | 0.3 |
| 4-Methoxyestrone | 4-MeO-E₁ | FIG. 2H | ND | ND | ND | ND | ND |
| 4-Methoxyestradiol | 4-MeO-E₂ | FIG. 2K | 708.1 | 2 | 891.3 | 1 | 2 |
| 4-Methoxyestriol | 4-MeO-E₃ | FIG. 2L | 1258.1 | 1 | 631.1 | 1 | 1 |
| 4-Bromoestradiol | 4-Br-E₂ | FIG. 3B | 158.5 | 7 | 25.1 | 35 | 0.2 |
| Estrone 3-sulfate | E₁-3-sulfate | FIG. 3L | ND | ND | ND | ND | ND |
| Estradiol 3-sulfate | E₂-3-sulfate | FIG. 3M | ND | ND | ND | ND | ND |

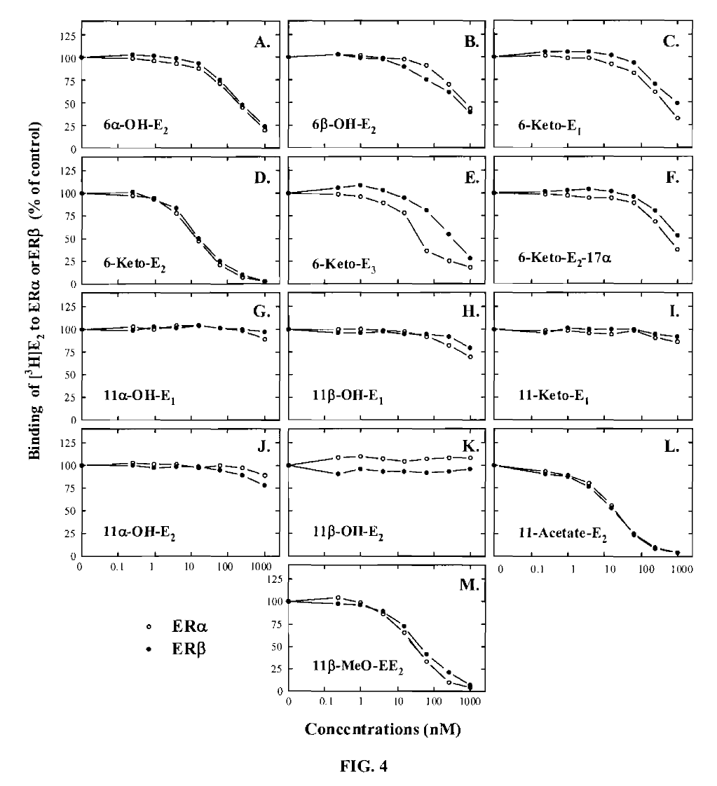

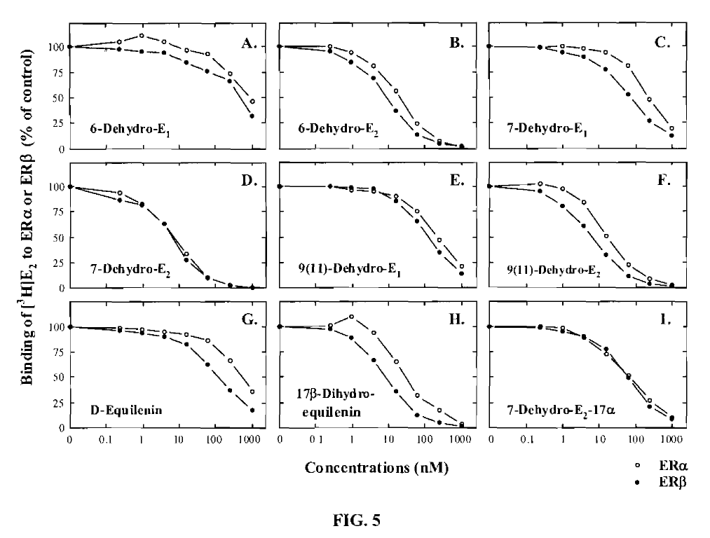

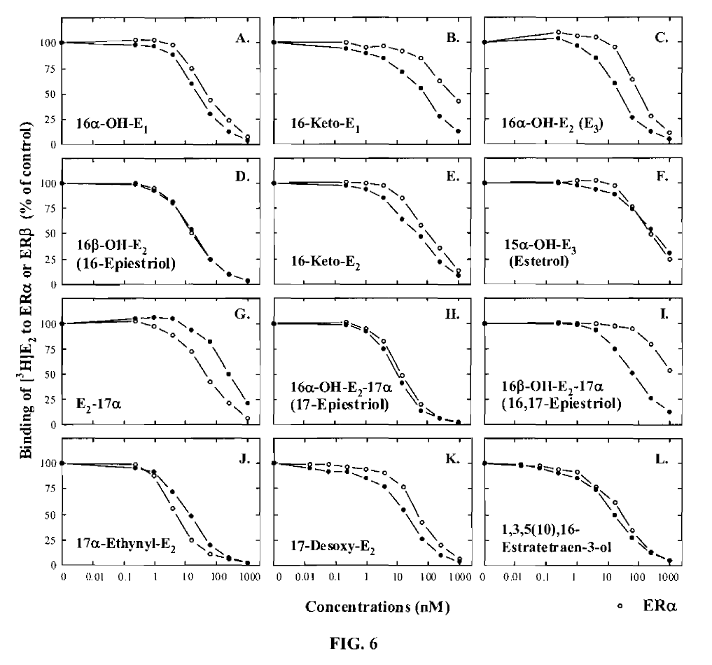

B-RING METABOLITES (C-6)

| | | | | | | | |
|---|---|---|---|---|---|---|---|
| 6-Ketoestrone | 6-Keto-E₁ | FIG. 4C | 489.8 | 2 | 891.3 | 1 | 2 |
| 6α-Hydroxyestradiol | 6α-OH-E₂ | FIG. 4A | 199.5 | 6 | 251.2 | 4 | 1.5 |
| 6β-Hydroxyestradiol | 6β-OH-E₂ | FIG. 4B | 794.3 | 1 | 562.3 | 2 | 0.5 |
| 6-Ketoestradiol | 6-Keto-E₂ | FIG. 4D | 15.9 | 71 | 15.9 | 56 | 1.3 |
| 6-Ketoestriol | 6-Keto-E₃ | FIG. 4E | 44.7 | 25 | 316.2 | 3 | 8.3 |
| 6-Ketoestradiol-17α | 6-Keto-E₂-17α | FIG. 4F | 562.3 | 2 | 1122.1 | 1 | 2 |
| 6-Dehydroestrone | 6-Dehydro-E₁ | FIG. 5A | 1000.1 | 1 | 501.2 | 2 | 0.5 |
| 6-Dehydroestradiol | 6-Dehydro-E₂ | FIG. 5B | 22.4 | 50 | 10.2 | 89 | 0.1 |
| 7-Dehydroestrone (Equilin) | 7-Dehydro-E₁ | FIG. 6C | 251.2 | 4 | 70.8 | 13 | 0.3 |
| 7-Dehydroestradiol (17β- | 7-Dehydro-E₂ | FIG. 6D | 7.9 | 142 | 7.9 | 113 | 1.3 |
| Dihydroequilin) | | | | | | | |
| 7-Dehydroestradiol-17α (17α- | 7-Dehydro-E₂-17α | FIG. 6I | 63.1 | 18 | 63.1 | 14 | 1.3 |
| Dihydroequilin) | | | | | | | |
| 9(11)-Dehydroestrone | 9(11)-Dehydro-E₁ | FIG. 6E | 223.9 | 5 | 141.3 | 6 | 0.8 |
| 9(11)-Dehydroestradiol | 9(11)-Dehydro-E₂ | FIG. 6F | 12.9 | 64 | 7.5 | 119 | 0.5 |
| D-Equilenin | D-Equilenin | FIG. 6G | 562.3 | 2 | 125.9 | 7 | 0.3 |
| 17β-Dihydroequilenin | 17β- | FIG. 6H | 31.6 | 35 | 8.9 | 100 | 0.4 |
| | Dihydroequilenin | | | | | | |

C-RING METABOLITES (C-11)

| | | | | | | | |
|---|---|---|---|---|---|---|---|
| 11α-Hydroxyestrone | 11α-OH-E₁ | FIG. 4G | ND | ND | ND | ND | ND |
| 11β-Hydroxyestrone | 11β-OH-E₁ | FIG. 4H | ND | ND | ND | ND | ND |
| 11-Ketoestrone | 11-Keto-E₁ | FIG. 4I | ND | ND | ND | ND | ND |
| 11α-Hydroxyestradiol | 11α-OH-E₂ | FIG. 4J | ND | ND | ND | ND | ND |
| 11β-Hydroxyestradiol | 11β-OH-E₂ | FIG. 4K | ND | ND | ND | ND | ND |
| 17β-Estradiol 11-acetate | 11-Acetate-E₂ | FIG. 4L | 20.1 | 56 | 19.9 | 45 | 1.2 |
| 11β-Methoxyethynylestradiol | 11β-MeO-EE₂ | FIG. 4M | 31.6 | 35 | 44.7 | 20 | 1.8 |

D-RING METABOLITES

| | | | | | | | |
|---|---|---|---|---|---|---|---|
| 15α-Hydroxyestriol (Estetrol) | 15α-OH-E₃ | FIG. 6F | 281.8 | 4 | 354.8 | 3 | 1.3 |
| 16α-Hydroxyestrone | 16α-OH-E₁ | FIG. 6A | 56.2 | 20 | 25.1 | 35 | 0.1 |
| 16-Ketoestrone | 16-Keto-E₁ | FIG. 6B | 631.1 | 2 | 89.1 | 10 | 0.2 |
| 16α-Hydroxyestradiol (Estriol) | 16α-OH-E₂ (E₃) | FIG. 1C | 100 | 11 | 25.1 | 35 | 0.3 |
| | | FIG. 6C | | | | | |
| 16β-Hydroxyestradiol (16- | 16β-OH-E₂ | FIG. 6D | 17.8 | 63 | 17.8 | 50 | 1.3 |
| Epiestriol) | | | | | | | |
| 16-Ketoestradiol | 16-Keto-E₂ | FIG. 6E | 112.2 | 1 | 50.1 | 18 | 0.1 |
| 16α-Hydroxyestradiol-17α (16, | 16α-OH-E₂-17α | FIG. 6H | 15.8 | 71 | 11.2 | 79 | 0.9 |
| 17-Epiestriol) | | | | | | | |
| 16β-Hydroxyestradiol-17α | 16β-OH-E₂-17α | FIG. 6I | 1258.9 | 1 | 70.8 | 13 | 0.1 |
| (16,17-Epiestriol) | | | | | | | |
| Estradiol-17α | E₂-17α | FIG. 1D | 50.1 | 22 | 281.8 | 3 | 7.3 |
| | | FIG. 6G | | | | | |
| 17α-Ethynylestradiol | 17α-EE₂ | FIG. 6J | 5.6 | 200 | 15.9 | 56 | 3.6 |
| 17-Desoxyestradiol | 17-Desoxy-E₂ | FIG. 6K | 70.8 | 16 | 19.9 | 45 | 0.4 |
| 1,3,5(10),16-Estratetraen-3-ol | 16-Estratetraen | FIG. 6L | 31.6 | 35 | 14.1 | 80 | 0.4 |

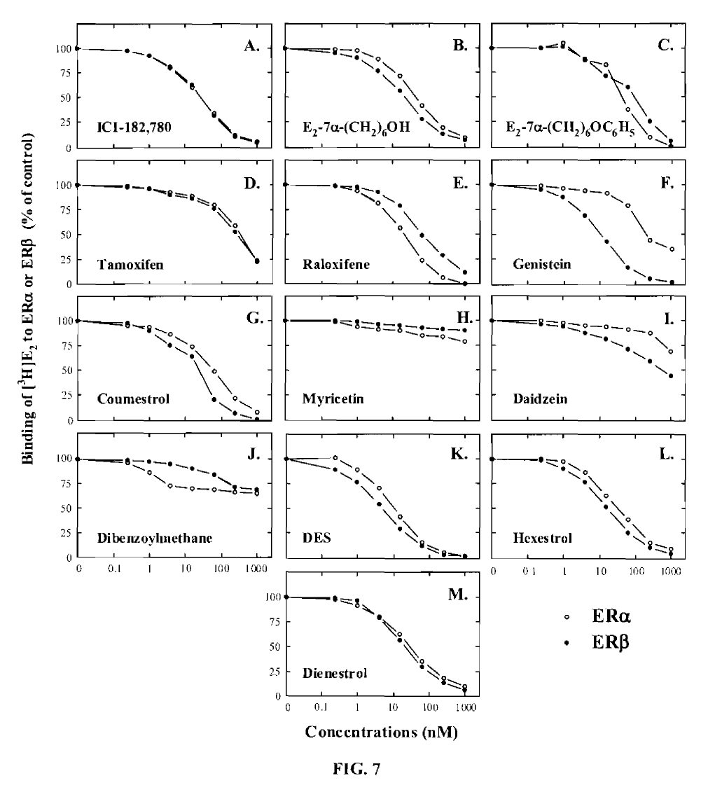

SOME OTHER ANALOGS

| | | | | | | | |
|---|---|---|---|---|---|---|---|
| ICI-182,780 | ICI-182,780 | FIG. 7A | 25.1 | 45 | 25.1 | 35 | 1.3 |
| 7α-(6-Hydroxyhexanyl)-17β- | E₂-7α-(CH₂)₆OH | FIG. 7B | 41.8 | 27 | 22.3 | 40 | 0.7 |
| estradiol | | | | | | | |
| 7α-(6-Benzyloxyhexanyl)-17β- | E₂-7α-(CH₂)₆OC₆H₅ | FIG. 7C | 39.8 | 28 | 63.1 | 14 | 2.0 |
| estradiol | | | | | | | |
| Tamoxifen | Tamoxifen | FIG. 7D | 354.8 | 3 | 251.2 | 4 | 0.8 |
| Raloxifene | Raloxifene | FIG. 7E | 22.4 | 50 | 63.6 | 14 | 3.6 |
| Genistein | Genistein | FIG. 7F | 199.5 | 6 | 11.2 | 79 | 0.1 |
| Coumestrol | Coumestrol | FIG. 7G | 50.2 | 22 | 25.1 | 35 | 0.6 |
| Myricetin | Myricetin | FIG. 7H | ND | ND | ND | ND | ND |
| Daidzein | Daidzein | FIG. 7I | 1000 | 0.1 | 631 | 2 | 0.05 |
| Dibenzoylmethane | DBM | FIG. 7J | ND | ND | ND | ND | ND |
| Diethylstilbestrol | DES | FIG. 7K | 11.2 | 100 | 5.4 | 166 | 0.6 |
| Dienestrol | Dienestrol | FIG. 7L | 30.2 | 37 | 19.95 | 56 | 0.7 |
| Hexestrol | Hexestrol | FIG. 7M | 36.3 | 31 | 18.62 | 60 | 0.5 |

The “ND” indicates that the corresponding IC₅₀ values could not be determined because the maximal inhibition of the receptor binding at the highest concentration tested (namely, 1000 nM) did not reach 50%. However, it will be appreciated that the RBA_{α}/RBA_{β} for some compounds may be determined by extrapolating the curves set forth in FIGS. 3-7. For example, it was determined that the RBA_{α}/RBA_{β} for 2-methoxyestrone, 2-nitorestrone, and 4-nitroestrone, was about 1, 3, and 10, respectively.

Binding Affinities of E₁, E₂, and E₃ for Human ERα and ERβ

E₁ (estrone), E₂ (estradiol-17β), and E₃ (estriol or 16α-OH-E₂) are three well-known human estrogens. Among all estrogens analyzed in this study, E₂ was found to have nearly the highest binding affinity for both ERα and ERβ, and its binding affinities for these two ER subtypes were very similar (FIG. 1A, Table 2). Using different concentrations of [³H]E₂ as ligand, the apparent K_{D} values was also determined for the recombinant human ERα and ERβ (FIG. 1E, 2F). Based on curve regression analysis of the receptor binding data, the K_{D} of E₂ for human ERα was 0.7 nM, and its K_{D} for ERβ was 0.75 nM. The values are slightly higher than some of the earlier measurements (average about 0.3 nM) using the crude ER protein preparations from various human tissues or cell lines. See Katzenellenbogen et al., In vivo and in vitro steroid receptor assays in the design of estrogen pharmaceuticals, In: Eckelman W C (editor), Receptor-Binding Radiotracers 1, CRC, Boca Raton, Fla., pp. 93-126 (1982); Fishman, Biological action of catecholestrogens, J. Endocr. 85, 59P-65P (1981). This difference likely was due to the relatively higher concentration of the recombinant ERα and ERβ proteins used in our in vitro receptor binding assays, and perhaps also due to the absence of other cellular proteins or components that usually partner the steroid receptors in subcellular crude extracts or in vivo.

E₁ had 10% of the binding affinity of E₂ for human ERα, and had 2% of the affinity of E₂ for ERβ (FIG. 1B, Table 2). E₃ also had markedly diminished binding affinity for ERα compared to E₂ (RBA 10% of E₂), but it had rather high binding affinity for ERβ (RBA 35% of E₂) (FIG. 1C, Table 2). For comparison, the binding affinity of E₂-17α (a C-17 isomeric analog of E₂) was determined for human ERα and ERβ. While E₂-17α retained considerable binding affinity for human ERα (RBA 22% of E₂), its binding affinity for ERβ was much lower (RBA only 3% of E₂) (FIG. 1D, Table 2). Notably, the relative binding affinities and binding preference of E₂-17α for human ERα and ERβ mirror those of E₁.

Notably, E₁ and E₃ are perhaps the two best known metabolites of E₂ in humans. Although these two endogenous E₂ derivatives had markedly lower binding affinities for human ERα and ERβ than E₂ (FIG. 1), it is of interest to point out that the facile metabolic conversion of E₂ to E₁ or of E₂ to E₃ in a woman may confer differential activation of the ERα or ERβ signaling system under different physiological conditions. For instance, E₁ had 4-fold higher relative binding affinity for human ERα than for ERβ, and this estrogen metabolite is present in larger quantities than E₂ in circulation as well as in most tissues of a non-pregnant woman, largely due to the actions of high levels of the oxidative 17β-hydroxysteroid dehydrogenase(s). Hence, the facile metabolic conversion of E₂ to E₁ would effectively produce a preferential activation of the ERα signaling system over the ERβ system in most target tissues of a non-pregnant woman. In contrast, E₃ has a more than five-fold preference for the activation of human ERβ over ERα, and it is a quantitatively-predominant estrogen metabolite produced during pregnancy. It is of interest to suggest that the very high levels of E₃ present during pregnancy may produce a differential activation of the ERβ signaling system in the pregnant woman and fetus for fulfilling various unique physiological functions.

A-Ring Metabolites.

Catechol Estrogens.

2-OH-E₂ is the most abundant hydroxy-E₂ metabolite formed in human liver. Largely because of its rather low estrogenic activity as measured earlier in laboratory animals (ovariectomized or immature rats or mice) and also in cultured human breast cancer cells, this catechol-E₂ metabolite was generally considered to have a very weak estrogenic activity in human. It has been widely accepted the notion that increased metabolic formation of 2-OH-E₂ in viva as opposed to the formation of other oxidative metabolites such as 4-OH-E₂, 16α-OH-E₁, or 16-OH-E₂ (E₃), would significantly reduce estrogen's hormonal activity in human and thus would be beneficial for the reduction of breast cancer risk. In the present experiments, 2-OH-E₂ had comparable binding affinity for ERα and ERβ, and its RBAs for ERα and ERβ were 22% and 35%, respectively, of E₂ (FIG. 2B, Table 2). The assays were repeated twice, and highly consistent results were obtained.

Despite its relatively high ER binding affinity, 2-OH-E₂ may still be a highly beneficial metabolite of E₂ in human owing to its rapid metabolic O-methylation in vivo which deactivates its hormonal activity and also concomitantly forms the anticarcinogenic 2-MeO-E₂. See Zhu et al., Functional role of estrogen metabolism in target cells: Review and perspectives, Carcinogenesis 19, 1-27 (1998); Zhu et al., Is 2-methoxyestradiol an endogenous estrogen metabolite that inhibits mammary carcinogenesis, Cancer Res. 58, 2269-2277 (1998). It is of note that although 2-OH-E₁ has relatively low binding affinity for human ERα and ERβ (significantly lower than that of 2-OH-E₂), this oxidative E₁ metabolite has a significant preference for binding to ERα over ERβ. Taking together the ER-binding data for E₁ and 2-OH-E₁, it is interesting to see that these two quantitatively-predominant estrogens normally present in non-pregnant woman would consistently produce a preferential activation of ERα over ERβ.

Different from 2-OH-E₂, 4-OH-E₂ is known to retain strong estrogenic activity and high ER binding affinity, and the data from this example also showed that this catechol-E₂ metabolite retained high and almost identical binding affinity for ERα and ERβ, with RBAs 70% and 56% of E₂, respectively (FIG. 2E, Table 2). In comparison, 2-OH-E₁ and 4-OH-E₁ (a quantitatively-minor metabolite) each had markedly weaker binding affinity for ERα and ERβ. While 4-OH-E₁ had almost identical binding affinity for ERα and ERβ (FIG. 2D), 2-OH-E₁ (the quantitatively-predominant endogenous oxidative metabolite of E₁) had a substantially higher affinity for ERα than for ERβ (FIG. 2A). 2-OH-E₃ had weak and similar binding affinity for ERα and ERβ (FIG. 2C).

### 2- or 4-Methoxyestrogens

All of the monomethylated catechol-E₁ metabolites tested in this study (2-MeO-E₁, 2-OH-3-MeO-E₁, and 4-MeO-E₁) did not have appreciable binding affinity for human ERα and ERβ at concentrations up to 1000 nM (FIGS. 3F, 3G, 3H). However, the two major monomethylated catechol-E₂ metabolites (2-MeO-E₂ and 4-MeO-E₂) each retained weak but similar binding affinities for both ERα and ERβ (FIGS. 2I, 3K and Table 2), with RBAs 1-2% of E₂. The estimated binding affinities are considerably higher than earlier measurements using cytosols prepared from human breast cancer. The weak ER-binding activity of 2-MeO-E₂ is believed to be mainly responsible for its moderate growth-stimulatory effect in ER-positive human breast cancer cells when exogenous estrogens were not present. See Liu et al., Concentration-dependent mitogenic and antiproliferative actions of 2-methoxyestradiol in estrogen receptor-positive human breast cancer cells, J. Steroid. Biochem. Mol. Biol. 88, 265-275 (2004). In comparison, 2-OH-3-MeO-E₂ (a close structural analog of 2-MeO-E₂) had a substantially weaker binding affinity for ERα and ERβ than 2-MeO-E₂ (FIG. 2J). 4-MeO-E₃ also retained weak but similar binding affinity for ERα and ERβ (FIG. 2L), and its affinity was comparable to those of 2-MeO-E₂ and 4-MeO-E₂.

2-Ethoxy-E₂ is an analog of 2-MeO-E₂ with strong anticancer activity (29, 30). See Wang et al., Synthesis of B-ring homologated estradiol analogues that modulate tubulin polymerization and microtubule stability, J. Med. Chem. 43, 2419-2429 (2000); Cushman et al., Synthesis, antitubulin and antimitotic activity, and cytotoxicity of analogs of 2-methoxyestradiol, an endogenous mammalian metabolite of estradiol that inhibits tubulin polymerization by binding to the colchicine binding site, J. Med. Chem. 38, 2041-2049 (1995). The compound retained a weak binding affinity for ERα and ERβ (FIG. 3E), and its affinity is slightly weaker than 2-MeO-E₂, probably due to the bulkier size of the ethoxy group at the C-2 position compared to a methoxy group.

Some Other A-Ring Analogs.

The binding affinities of several semi-synthetic A-ring derivatives of E₂ (data shown in FIG. 3) were also compared. Notably, some earlier studies have suggested that substitution of small functional groups at the C-2 and C-4 positions are reasonably well tolerated, whereas larger groups may readily reduce ER binding affinity because they may involve the formation of an intra-molecular hydrogen bond with the C-3 hydroxyl group. See Anstead et al., The estradiol pharmacophore: ligand structure-estrogen receptor binding affinity relationships and a model for the receptor binding site, Steroids 62, 268-303 (1997). However, it was observed that, in some cases, substitution of even a very small group such as bromine at the C-2 position of E₂ (2-Br-E₂) drastically reduced its binding affinity for ERβ (RBA only <0.5% of E₂), while this substitution reduced its binding affinity for ERα to a relatively lesser degree (RBA 4% of E₂)) (FIG. 3A). This observation is rather interesting since C-2 bromine substitution had a far stronger negative effect on ER binding (particularly for ERβ) than the C-2 hydroxyl substitution. Notably, bromine substitution at the C-4 position of E₂ (4-Br-E₂) affected its binding affinity for ERα and ERβ in an opposite manner as what was observed for 2-Br-E₂. The 4-Br-E₂ compound had a decreased binding affinity for ERα about 5 times more than for ERβ. Similarly, addition of a methyl group to the C-4 position of E₂ (4-Methyl-E₂) also decreased its binding affinity for ERα (RBA only 7% of E₂) more than for ERA (RBA 35% of E₂), See FIGS. 3B and 3D.

Addition of a methyl group to the C-1 position of E₂ (1-Methyl-E₂) decreased its binding affinity to a similar degree (by approximately 90%) for ERα and ERβ (FIG. 3C). Several earlier studies have also shown that the C-1 substitution of E₂ (regardless of polarity of the substituents) all had a negative effect on the binding affinity for crude ER proteins from rabbit or human. See Anstead et al., The estradiol pharmacophore: ligand structure-estrogen receptor binding affinity relationships and a model for the receptor binding site, Steroids 62, 268-303 (1997). This influence was thought to be due to a direct interaction of the C-1 substituting group with the ER protein rather than a structural perturbation of the ligand conformations.

The 2,3-dimethylated catechol E₁ and E₂ derivatives did not have any appreciable binding affinity for ERα and ERβ (FIG. 3F, 4G). This is in accord with earlier studies using the rat uterine ER protein preparations. See Ball et al., Calecholoestrogens (2- and 4-hydroxyoestrogens): Chemistry, biogenesis, metabolism, occurrence and physiological significance, Acta. Endocrinol. 232, 1-127 (1980).

As expected, several synthetic C-2 or C-4 substitution analogs containing an amino (—NH₂) or nitro (—NO₂) group resulted in diminished its binding affinity for ERα and ERβ (FIG. 3H-K). In particular, E₁ (2-NH₂-E₁, 2-NO₂-E₁, 4-NH₂-E₁ and 4-NO₂-E₁) derivatives only retained very weak binding affinities for human ERα and ERβ. It is of note that the —NO₂ and —NH₂ substitutions of E₁ produced inhibition curves with rather shallow slopes, which likely suggests that these were not pure competitive inhibition.

The C-3 sulfated estrogens (E₁-3-sulfate and E₂-3-sulfate) were found to be basically devoid of appreciable binding affinity for ERα and ERβ (FIG. 3L, 3M), which was consistent with earlier findings. Like the C-3 sulfated estrogens, earlier studies have shown that E₂ 3-ethyl ether (43, 44) or 2-desoxy-E₂ (see Fanchenko et al., The specificity of human estrogen receptor, Acta. Endocrinol. 7, 232-240 (1979); and Brooks et al., Estrogen structure-receptor function relationships, Moudgil V K (ed.), Recent Advances in Steroid Hormone Action, Walter de Gruyter, Berlin, pp. 443-466 (1987)), each had very low binding affinity for ER compared to E₂. It was suggested that the C-3 hydroxyl group of E₂ functions primarily as an H-bond donor in its interactions with ERα and ERβ. According to more recent x-ray crystallography study of the human ERα and ERβ bound with E₂, it appears that the very low binding affinities of various C-3 modified E₂ derivatives are due to a combination of disturbance of H-bond formation and steric hindrance.

B-Ring and C-Ring Metabolites.

C-6 Substituted Estrogens.

The data shows that addition of a hydroxyl group to the C-6α or C-6β position of E₂ markedly reduced its binding affinity for both ERα and ERβ, but addition of a keto group to the C-6 positions of E₂ or E₁ did not significantly affect the original binding affinity of these estrogens for ERα or ERβ.

Among six B-ring hydroxylated or keto metabolites of E₂ or E₁ tested in this study, all of them retained certain degrees of binding affinity for both ERα and ERβ (FIG. 4A-4F and Table 2). 6α-OH-E₂ or 6β-OH-E₂ had markedly reduced binding affinities for ERα and ERβ compared to E₂ (FIG. 4A, 4B). However, addition of a keto group to the C-6 position of E₂ did not markedly affect its original binding affinity for ERα and ERβ(FIG. 4C). In comparison, addition of a C-6 keto group to E₁ differentially altered its binding affinity for ERα and ERβ (RBAs 23% and 50% of E₁, respectively) (FIG. 4D, Table 2). Similarly, addition of a C-6 keto to E₂-17α (6-keto-E₂-17α) also markedly reduced its binding affinity for ERα (RBA 9% of E₂-17α), but its binding affinity for ERβ was decreased to a relatively lesser extent (RBA 25% of E₂-17α) (FIG. 4F, Table 2). However, addition of a C-6 keto group to E₃ slightly increased its binding affinity for ERα (RBA 224% of E₃), but it drastically reduced its binding affinity for ERβ (RBA 8% of E₃) (FIG. 4E, Table 2).

C-11 Substituted Estrogens.

The data with the C-11 position derivatives were rather interesting and revealing. Addition of a hydrophilic group (such as a hydroxyl or keto group) to the C-11 position of E₂ or E₁ almost completely abolished their binding affinities for both ERα and ERβ. This was true regardless of whether the substitution was 11α or 11β (FIG. 4G-4J). However, substitution of a lipophilic group with even a bulkier size (such as the acetate or methoxy group) did not significantly affect the binding affinity for either ERα or ERβ (FIG. 4L, 4M, Table 2). These data indicated that the drastic decrease in the binding affinities of 11α-OH-E₂, 11-OH-E₂, or 11-keto-E₂ for human ERα and ERβ is not due to steric hindrance caused by the C-11 position substitutions, but it is primarily due to alterations of the lipophilicity near the C-11 position. It is of note that earlier studies have also shown that the C-11β position of E₂ was tolerant of even very large substituents, if the polar functional groups were placed at a distance from the steroid core structure (reviewed in ref. Anstead et al., The estradiol pharmacophore: ligand structure-estrogen receptor binding affinity relationships and a model for the receptor binding site, Steroids 62, 268-303 (1997); and Gao et al., Comparative QSAR analysis of estrogen receptor ligands, Chem. Rev. 99, 723-744 (1999)). These observations agree well with recent homology modeling data for human ERα and ERβ showing that there is considerable space near the C-7α binding site of E₂ which can readily accommodate various estrogen analogs with a rather bulky/lengthy substitution (data not shown).

Dehydroestrogens.

In addition to the B-ring and C-ring substitution metabolites described above, this example also investigated several common B- or C-ring dehydrogenated E₂ or E₁ metabolites. It was found that found that most of the dehydroestrogen metabolites retained rather high binding affinities for both ERα and ERβ compared to their respective non-dehydrogenated precursors, and some of them [such as 6-dehydro-E₂ and 9(11)-dehydro-E₂] retained high binding affinities for human ERs. The data summarized in FIG. 5 and Table 2. More specifically, 6-dehydro-E₂ and 9(11)-dehydro-E₂ had similar or somewhat higher binding affinity for human ERβ compared to E₂ (RBAs 89% and 119%, respectively, of E₂), but their binding affinities for ERα were slightly reduced, with RBAs 50% and 64% of E₂, respectively (FIG. 5B, 5F, Table 2).

Notably, several of the dehydrogenated estrogens tested in this study are the major components (in their conjugated forms) present in Premarin®, the commonly-used hormone replacement therapy in peri-menopausal and post-menopausal women. As discussed more fully below, the main estrogenic ingredients include sodium E₁ sulfate, sodium equaling sulfate, and the sodium sulfate conjugates of E₂-17α, 17α-dihydroequilenin, and 17β-dihydroequilin. Equilin (7-Dehyro-E₁) and 9(11)-dehydro-E₁ each had slightly decreased binding affinity for ERα compared to E₁ (RBAs 45% and 50% of E₁, respectively), but they had a drastically increased binding affinity for ERβ (RBAs 631% and 316% of E₁, respectively).

The binding affinities of 17β-dihydroequilin (i.e., 7-dehydro-E₂) for human ERα and ERβ were actually slightly higher than E₂ (its RBAs 142% and 113%, respectively, of E₂) (FIG. 5D). Similarly, while the binding affinity of 17α-dihydroequilin (i.e., 7-dehydro-E₂-17α) for ERα remained the about same as that of E₂-17α, this equine estrogen had a more than fourfold higher binding affinity for ERβ than E₂-17α (RBA 447% of E₂-17α) (FIG. 5I, Table 2). Compared to E₁, 6-dehydro-E₁ had nearly the same binding affinity for ERβ, but its binding affinity for ERα was significantly decreased, with its RBA only 10% of E₁ (FIG. 5A, Table 2).

D-Equilenin had a much weaker binding affinity than E₁ for human ERα (RBA 20% of E₁), but its binding affinity for ERβ was more than 3 times higher than that of E₁. Very similarly, while 17β-dihydroequilenin had a low binding affinity for ERα (35% of E₂), it had a high binding affinity for ERβ (RBA 100% of E₂) (FIG. 5H). Taken together, it is evident that many of the equine estrogens contained in Premarin have a differential binding affinity for human ERβ over ERα.

D-Ring Metabolites.

A total of twelve D-ring metabolites/derivatives of E₂ and E₁ were studied (data summarized in FIG. 6A-6L, Table 2). The data showed that E₁ only had 5-10% of the binding affinity of E₂ for human ERα and ERβ, and it had a significant preference for binding to ERα. The markedly reduced binding affinity of E₁ for ERs has previously been suggested to reflect the unique importance of the C-17β hydroxyl in enhancing its interactions with the ER molecules. This suggestion was also supported by other studies showing that when the C-17β hydroxyl of E₂ was converted to a methyl ether or an acetate, their ER binding affinities were greatly diminished. See Katzenellenbogen et al., Photoaffinity labels for estrogen binding proteins of rat uterus, Biochemistry 12, 4085-4092 (1973); Kaspar et al., Shielding effets at 17α-substituted estrogens. A tentative explanation for the low biological activity of 17α-ethyl-estradiol based on IR and NMR spectroscopic studies, J. Steroid. Biochem. 23, 611-616 (1985). In this example, the data also showed that when the entire C-17β hydroxyl group was absent, the derivatives [i.e., 17-desoxy-E₂ and 1,3,5(10),16-estratetraen-3-ol] actually had quite high binding affinity for ERα and ERβ, which was much higher than that of E₁, but lower than E₂ (FIG. 6, Table 2). The data are also consistent with a few earlier reports on the binding affinity of 17-desoxy-E₂ for human and rat estrogen receptors. See Fanchenko et al., The specificity of human estrogen receptor, Acta. Endocrinol. 7, 232-240 (1979); Brooks et al., Estrogen structure-receptor function relationships, Moudgil V K (ed.), Recent Advances in Steroid Hormone Action, Walter de Gruyter, Berlin, pp. 443-466 (1987). Taking together all the information we have gathered, it appears that while the presence of the C-17β hydroxyl group (but not a C-17α hydroxyl or C-17 keto group) increases the binding affinity of an aromatic steroid for human ERα and ERβ, its relative influence likely is not as strong as that of the C-3 hydroxyl group.

The binding affinity of 16α-OH-E₁ for human ERα was twice as high as that of E₁, but its affinity for ERβ was 18-fold higher than E₁ (FIG. 6A). Further, its binding affinity was still lower than that of E₂ (with RBAs 56% and 25%, respectively, of E₂). This is one of the most notable eases that hydroxylation of an endogenous estrogen markedly enhanced its binding affinity for human ERα and/or ERβ than the respective parent hormone. In addition, an earlier study reported that this E₁ metabolite may be able to bind covalently to the ER protein through the formation of a Schiff's base, likely resulting in sustained ER-mediated growth stimulation of the target cells. These biochemical properties of 16α-OH-E₁ have been the basis for the well-known hypothesis that increased metabolic formation of 16α-OH-E₁ in a woman may increase the risk for development of estrogen-inducible cancers. Notably, despite its much higher binding affinities than those of E₁ for human ERα and ERβ, they were still slightly lower than E₂ (with RBAs of 56% and 25%, respectively, of E₂).

Interestingly, while 16-keto-E₁ only had 18% of the binding affinity of E₁ for ERα, its binding affinity for ERβ was five-fold higher than that of Et (RBA 501% of E₁) (FIG. 6B). Thus, the relative preference of 16-keto-E₁ for human ERβ over ERα is approximately 25 times higher than E₁. Addition of C-16 keto group to E₁ increased its binding affinity for ERβ but decreased its binding affinity for ERα.

Addition of a C-16 keto or a C-15α hydroxyl to E₂ each significantly decreased the binding affinity for ERα and ERβ compared to E₂. 16-Keto-E₂ and 15α-OH-E₃ (estetrol) each had a reduced binding affinity for both ERα and ERβ compared to E₂ and E₃, respectively (FIG. 6E, 6F)

As Already Mentioned Earlier, E₃ (Estriol 16α-OH-E₂), a Major D-Ring Metabolite in humans (particularly during pregnancy), had a markedly decreased binding affinity for ERα compared to E₂ (RBA 11% of E₂), but it retained a rather high binding affinity for ERβ (RBA 35% of E₂) (FIG. 6C, Table 2). By contrast, substitution of a C-16β hydroxyl group to E₂ (namely, 16β,17β-OH-E₂, also called 16-epiestriol) did not noticeably affect its binding affinity for either ERα or ERβ (FIG. 6D).

As already mentioned earlier, E₂-17α retained considerable binding affinity for ERα (RBA 22% of E₂), but it had substantially lower binding affinity for ERβ (3% of E₂) (FIG. 1D or 7G). Interestingly, addition of a hydroxyl group to the C-16α or C-16β position of E₂-17α affected its binding affinity for ERα and ERβ rather differently (FIG. 6H-6I). 16α-OH-E₂-17α (17-epiestriol) had very high, almost identical binding affinities for both ERα and ERβ (RBAs 71% and 79%, respectively, of E₂), which were 3 and 16 times higher, respectively, than its precursor E₂-17α. However, 16β-OH-E₂-17α (16,17-epiestriol) had low binding affinity for ERα, preferential binding affinity for ERβ over ERα, and the difference in the binding affinities is 18-fold.

17α-Ethynylestradiol (17α-EE₂), a semi-synthetic steroidal estrogen commonly used as an estrogenic component in various oral contraceptives, had very high binding affinity for both ERα and ERβ compared to E₂. The binding affinity of 17α-EE₂ for ERα was twice as high as that of E₂, but its affinity for ERβ was only about half of that of E₂ (Table 2 and FIG. 6J). The same receptor binding assay with this estrogen was repeated twice, and consistent results were obtained. Accordingly, the relative ratio of preference for binding to ERα and ERβ by 17α-EE₂ is approximately 4 times of that for E₂. Interestingly, the removal of the C-17 hydroxyl group from E₂ (17-desoxy-E₂) did not drastically reduce its binding affinity for human ERα and ERβ (FIG. 6K). Similarly, 1,3,5(10),16-estratetraen-3-ol, which is also without a C-17 substitution, had a very similar binding affinity as that of 17-desoxy-E for ERα and ERβ (FIG. 6L).

It is worth noting that the 16α-hydroxylated estrogens (16α-OH-E₁ and E₃), epiestriols (16α-OH-E₂-17α, 16β-OH-E₂, and 16β-OH-E₂-17α), and other C-16 metabolites (e.g., 16-keto-E₁) are usually quantitatively-minor estrogen metabolites in non-pregnant woman, but some of them are formed in unusually large quantities during pregnancy, particularly at late stages of pregnancy. The data from this example revealed that many of these estrogen metabolites (e.g., E₃, 16β-OH-E₂-17α) had high preferential binding affinities for human ERβ over ERα. It is possible that they may jointly serve as important endogenous ligands for the preferential activation of the ERβ signaling pathway during human pregnancy. Such a preferential activation of ERβ may play an indispensable role in mediating the various actions of the endogenous estrogens required for the development of the fetus as well as for fulfilling other physiological functions of pregnancy. This suggestion is in line with some of the observations showing that the ERβ has a wide distribution in maternal rat reproductive organs as well as the fetus.

Based on all of the endogenous estrogen metabolites/derivatives analyzed in this example, it is apparent that the D-ring (particularly at the C-16 and C-17 positions) of E₂ is the most sensitive target where modifications of its structure may differentially modify its binding affinity for the human ERα or ERβ. This property will have important physiological as well as pharmacological implications. From a physiological point of view, it is known that E₂ is perhaps the most potent endogenous estrogen which has similar binding affinity for ERα and ERβ, but it is not the predominant estrogen(s) present in the body. In non-pregnant woman, the predominant form of estrogens in various tissues is E₁ (which has a higher ERα activity over ERβ), whereas in a pregnant woman, E₃ along with several other D-ring metabolites become the quantitatively-predominant forms of estrogens (which have strong preference for ERβ). From a pharmacological point of view, selective modifications of the D-ring of a steroidal estrogen may represent an efficient strategy for the rational design of selective/preferential agonists or antagonists for human ERα and particularly for ERβ.

In summary, most of the D-ring metabolites retained rather high binding affinity for human ERα and ERβ, but several of them (16β-OH-E₂-17α, 16α-OH-E₂-17α, 16-keto-E₁, 16α-OH-E₂, and 16α-OH-E₁) had markedly increased binding affinity for human ERβ over ERα compared to their respective precursors (namely, E₁, E₂, and E₂-17α).

Antiestrogens, Phytoestrogens, and Stilbene Estrogens.

For the purpose of comparison, the binding affinities of a number of steroidal and nonsteroidal antiestrogens, phytoestrogens, stilbene estrogens and nonaromatic steroids for human ERα and ERβ were also determined. Their data were summarized in FIG. 7A-M.

Steroidal and Nonsteroidal Antiestrogens.

The binding affinities of ICI-182,780 for both ERα and ERβ were very high and nearly the same (RBAs 45% and 35%, respectively, of E₂) (FIG. 7A and Table 2). Similarly, another two synthetic C-7α substituted analogs, E₂-7α-(CH₂)₆OH and E₂-7α-(CH₂)₆OC₆H₅, which have shorter side chains at the C-7α position than ICI-182,780, retained high and similar binding affinities for ERα and ERβ as the ICI compound (FIG. 7C, Table 2). This data is in agreement with the earlier suggestion that the human ER is tolerant of large/lengthy substitution at the C-7α position of E₂, if the polar group is placed away from the steroid core. Ongoing homology modeling studies of the binding of various bioactive estrogen derivatives with human ERα and ERβ also showed that there is considerable space near E₂'s C-7α-binding pocket which can accommodate ligands with a bulky substitution. Since the C-7α-binding position is mainly composed of lipophilic amino acid residues, this also explains that polar groups need to be placed away from the C-7α position in order to retain a high binding affinity with the receptor.

Tamoxifen and raloxifene are two well-known nonsteroidal ER antagonists (partial agonists). Tamoxifen had almost identical binding affinities for human ERα and ERβ (FIG. 7D), although its binding affinities for these two receptors were only 3-4% of those of E₂ and 7-10% of ICI-182,780.

In comparison, while raloxifene had a similar binding affinity for ERβ as tamoxifen, the former had 16-fold higher binding affinity for ERα than the latter and was comparable to ICI-182,780 (FIG. 7E, Table 2). Therefore, raloxifene actually had a strong preferential binding affinity for human ERα than for ERβ. Since raloxifene and tamoxifen are very different from each other in that the former had a strong preferential binding affinity for ERα, this may be one of the important underlying factors that determine their different pharmacological profiles in various target tissues. In addition, it is possible that differences in their metabolic conversion to derivatives with differing ER-binding affinities may also contribute to some of the known pharmacological differences of these two antiestrogens in vivo.

Phytoestrogens.

Genistein, a well-known phytoestrogen abundantly present in soy products, had an extremely high binding affinity for ERβ (almost identical to that of the endogenous hormone E₂), but its binding affinity for ERα was only 6% of its binding affinity for ERβ. This data is consistent with earlier reports. If one assumes that a significant portion of the ingested genistein is subsequently uptaken into target cells without degradation, then the practice of using dietary phytoestrogens (e.g., genistein) as the sole or main source of estrogens for female hormone replacement therapy may unwittingly confer a long-term predominant ERβ stimulation in postmenopausal women. Before the health benefits or potential side effects associated with a long-term ERβ stimulation in peri-menopausal or postmenopausal women are known, it may be risky to use dietary phytoestrogens as the sole or main source of estrogens for female hormone replacement therapy. Likewise, more studies are urgently needed to determine if there are any potential side effects in newborns or infants who feed entirely on soymilk (rich in genistein) instead of human or cow milk.

Coumestrol, another well-known phytoestrogen, had very high binding affinity for human ERα and ERβ, and its relative binding affinity for ERβ was slightly higher than its affinity for ERα (FIG. 7G). Myricetin basically had no appreciable binding affinity for human ERα and ERβ (FIG. 7H). Daidzein had very weak binding affinities for both ERα and ERβ, but its relative affinity for ERβ was significantly higher than its affinity for ERα (FIG. 7I). Dibenzoylmethane (DBM) had a weak overall binding affinity for ERα and ERβ (FIG. 7J).

Stilbene Estrogens.

Many earlier animal studies as well as in vitro receptor binding assays have shown that diethylstilbesterol (DES), dienestrol, and hexestrol are very potent synthetic estrogens with similar estrogenic potency and efficacy as E₂. The results from this example also showed that each of these stilbene estrogens had very high binding affinity (similar to that of E₂) for both human ERα and ERβ. The three well-known non-steroidal stilbene estrogens (diethylstilbesterol [DES], dienestrol, and hexestrol) had very high binding affinities (similar to that of E₂) for human ERα and ERβ (FIG. 7K-M, Table 2). We noted that DES and hexestrol had a slightly higher binding affinity for ERβ than for ERα, although the difference was only very small.

In this example, the activity of a large number of endogenous estrogen metabolites, including those contained in Premarin®, for human ERα and ERβ was investigated. It was found that while E₂ (perhaps the best-known endogenous estrogen) has nearly the highest and almost identical binding affinities for human ERα and ERβ, many of its metabolites have widely different preference for the activation of human ERα and ERβ. It is of particular interest to note that the predominant estrogens that are present in a pregnant woman are very different from those present in a non-pregnant woman. Furthermore, these estrogens have widely different preference for activation of human ERα and ERβ.

Many of the endogenous estrogen metabolites retained varying degrees of similar binding affinity for ERα and ERβ, but some of them retained differential binding affinity for the two subtypes. For instance, several of the D-ring metabolites, such as 16α-OH-E₂, 16β-OH-E₂-17α, and 16-keto-E₁, had distinct, preferential binding affinity for human ERβ over ERα (difference up to 18-fold). Notably, while E₂ has nearly the highest and equal binding affinity for ERα and ERβ, E₁ and 2-OH-E₁ (two quantitatively-predominant endogenous estrogens in non-pregnant woman) have preferential binding affinity for ERα over ERβ, whereas 16α-OH-E₂ (estriol) and other D-ring metabolites (quantitatively-predominant endogenous estrogens formed during pregnancy) have preferential binding affinity for ERβ over ERα.

### Example 2

### Comparison of Endogenous Estrogens in Pregnant and Non-Pregnant Women

A large number of endogenous estrogen derivatives are known to be present in humans. In this example, the human urinary excretion of various estrogens (mostly as conjugates) as a global indicator of the biosynthesis and metabolism of endogenous estrogens in vivo was investigated. It is estimated that the total daily amount of various urinary estrogens excreted from a late pregnant woman is about 300 times higher than the amount excreted by a non-pregnant woman of the same age group. In addition, the composition of the urinary estrogens in women are also widely different. Representative profiles of various endogenous estrogens found in the urine of pregnant and non-pregnant young women are summarized in Table 3.

TABLE 3
The levels of endogenous estrogen metabolites present in the
urine samples from pregnant and non-pregnant women.

| | | | | | | Pregnant woman | |
|---|---|---|---|---|---|---|---|
| | | | | | | (ng/mL) | |
| | Non-pregnant woman (μg/24 h) | | | | | a month before | |
| | Day 6-10 | | Day 16 | Day 21-25 | | delivery | |
| | Average | S.D. | (peak) | Average | SD | Average | SD |
| E₁ (estrone) | 3.40 | 0.81 | 11.13 | 3.12 | 0.27 | 39.9 | 6.1 |
| E₂ (17β-estradiol) | 1.44 | 0.54 | 4.17 | 1.64 | 0.16 | 20.4 | 3.3 |
| 2-OH-E₁ | 6.06 | 1.70 | 42.74 | 11.13 | 3.27 | 13.4 | 2.5 |
| 4-OH-E₁ | 2.20 | 1.26 | 5.40 | 1.75 | 0.69 | 19.6 | 1.9 |
| 16α-OH-E₁ | 3.15 | 1.61 | 21.18 | 3.91 | 1.55 | 1358.6 | 210.3 |
| 2-MeO-E₁ | ND | ND | 7.38 | ND | ND | 12.0 | 5.1 |
| 2-OH-E₂ | 0.71 | 0.22 | 3.59 | 1.09 | 0.23 | 3.0 | 0.7 |
| 4-OH-E₂ | 0.69 | 0.09 | 1.42 | 0.95 | 0.26 | ND | ND |
| 2-MeO-E₂ | 0.97 | 0.38 | 1.40 | 0.77 | 0.29 | 3.4 | 1.2 |
| E₃ (estriol or 16α-OH-E₂) | 4.28 | 1.29 | 22.32 | 3.61 | 0.44 | 8177.5 | 763.1 |
| 16-EpiE₃ | ND | ND | ND | ND | ND | 45.9 | 7.6 |
| 17-EpiE₃ | 2.96 | 0.48 | 4.69 | 3.43 | 1.21 | 17.3 | 6.4 |
| 16,17-EpiE₃ | ND | ND | ND | ND | ND | 55.5 | 9.5 |
| 2-OH-E₃ | ND | ND | ND | ND | ND | 7.9 | 1.9 |
| 15α-OH-E₃ (estetrol) | ND | ND | ND | ND | ND | 30.7 | 6.5 |

*Twenty-four hour urine sample from pregnant women was not available, and the data is organized in the concentration of ng estrogen metabolite/mL urine.
*It is possible that the estrogen concentrations in the urine were increased to higher levels before delivery, but the concentrations of estrogens in urine returned to normal levels very quickly after delivery.

In the urine samples obtained from non-pregnant young women, the conjugated forms of 2-hydroxy-estrone, followed by 16α-hydroxy-estradiol (E₃), 16α-hydroxyestrone, and estrone (E₁), are the predominant estrogens. The amount of E₂ and its major metabolites 2-hydroxy-estradiol and 2-methoxy-estradiol was much less than that of estrone and its corresponding metabolites. The relative composition of various estrogens in circulation is believed to be largely comparable to what is seen in the urine. The presence of higher levels of estrone (E₁) over estradiol (E₂) in a non-pregnant woman is largely attributable to the high levels of the oxidative 17β-hydroxysteroid dehydrogenase (“17β-HSD”), which catalyzes the facile conversion of estradiol (E₂) to estrone (E₁). The conversion of estrone (E₁) to 2-hydroxy-estrone or estradiol (E₂) to 2-hydroxy-estradiol is catalyzed by various cytochrome P450 enzymes, and the subsequent O-methylation to form 2-methoxy-estrone or 2-methoxy-estradiol is catalyzed by catechol-O-methyltransferase (“COMT”).

There is a drastic change in the endogenous estrogen composition during pregnancy. Estriol (E₃) becomes the predominant estrogen and it is produced in unusually large quantities. The daily amount of this estrogen (in its conjugated forms) released into the urine of a late pregnant woman is 200-1000 times higher than any of the quantitatively-major estrogens produced in a non-pregnant woman. Notably, several other D-ring estrogen derivatives, such as 17-epi-E₃, 16-epi-E₃, 16,17-epi-E₃ and estetrol (15α-hydroxy-estriol), are also produced in readily detectable quantities at late stages of pregnancy. These D-ring derivatives are usually only present at low or undetectable levels in non-pregnant young women.

From Example 1, it was found that E₁ and 2-OH-E₁, two of the quantitatively-major estrogen derivatives present in a non-pregnant woman, have a modest but significant preference for binding to human ERα over ERβ. More specifically, E₁ had five-fold higher relative binding affinity for human ERα than for ERβ. Similarly, 2-OH-E₁ (the 2-hydroxylated metabolite of E₁) also has a about ten-fold preference for activation of ERα over ERβ.

Notably, E₁ and 2-OH-E₁ have markedly lower binding affinity for human ERα and ERβ compared to E₂. In the present invention, it is theorized that the relatively lower binding affinity of E₁ and 2-OH-E₁ is an advantage rather than a disadvantage, because such compounds would pose a lower risk for causing over-stimulation of the ERα and ERβ signaling systems in vivo.

In contrast, E₃, the quantitatively-predominant estrogen produced during pregnancy, has a significant preference for binding to ERβ over ERα. E₃ had a rather low binding affinity for human ERα compared to E₂ (RBA 11% of E₂), but it retained a relatively high binding affinity for ERβ (RBA 35% of E₂). Similarly, 16α-OH-E₁, another well-known hydroxy-E₁ metabolite that is also formed in increased quantity during pregnancy, has a higher binding affinity than E₁ for both ERα and ERβ.

16,17-Epiestriol, another estrogen in pregnant women, had a very low binding affinity for human ERα, but it had a preferential affinity for ERβ. The difference of its binding affinity for ERβ over ERα is 18-fold. The relative quantity of this estrogen in pregnant woman's urine is rather small (Table 3).

In sum, although 17β-estradiol (E₂) is perhaps the best-known endogenous estrogen in humans, it is not the predominant estrogen produced in the body of a pregnant woman or of a non-pregnant woman. Based on the information discussed above, it is evident that the major endogenous estrogens that are produced in a non-pregnant woman are vastly different in composition and quantity from those produced in a pregnant woman, Further, it is evident that there is a marked difference in the ratio and also intensity of ERα and ERβ activation in a non-pregnant young woman compared to a pregnant woman. The major estrogens produced in a non-pregnant woman would modestly favor the activation of the ERα system over the ERβ system. However, during pregnancy, there is a preponderance of activation of ERβ over ERα, which is exerted by various pregnancy estrogens, mainly estriol, which is produced in unusually large quantities. Such a preferential activation of ERβ is believed to play an indispensable role in mediating the various actions of endogenous estrogens that are required for the development of the fetus as well as for fulfilling other physiological functions related to pregnancy. This suggestion is in line with some of the observations showing that the ERβ has a wide distribution in maternal reproductive organs in rats as well as their fetus.

### Comparative Example 3

### Analysis of Premarin®

Premarin®, the commonly-used hormone replacement therapy, contains a mixture of conjugated estrogens obtained from pregnant mare's urine. As shown in the following table, the major estrogens produced in a pregnant mare are quite different from those produced in a pregnant woman.

TABLE 4
Composition of Premarin ®.

| Sodium estrogen sulfate | Mg/Tablet | ERα RBA | ERβ RBA | Ratio |
|---|---|---|---|---|
| Estrone (E₁) | 0.370 | 10 | 2 | 5 |
| 7-dehydroestrone (Equilin) | 0.168 | 4 | 13 | 0.3 |
| 17α-Dihydroequilin | 0.102 | 18 | 14 | 1.3 |
| 17α-Estradiol | 0.027 | 22 | 3 | 7.3 |
| 17β-Dihydroequilin | 0.011 | 142 | 113 | 1.3 |
| 17α-Dihydroequilenin | 0.021 | | | |
| Equilenin | 0.015 | 2 | 7 | 0.3 |
| 17β-Estradiol | 0.005 | 100 | 100 | 1 |
| Δ8,9-Dehydroestrone | 0.026 | | | |

The exact total amount of various estrogenic components contained in each Premarin® tablet is not known. It has been traditionally assumed to each Premarin® tablet contained a mixture of estrogen sulfates that are biologically equivalent to 0.625 mg of estrone 3-sulfate, according to an earlier uterotropic assay using ovariectomized female rats. Further, a synthetic Premarin® formulation is set forth in Hill, U.S. Pat. No. 6,855,703, which is incorporated by reference.

Table 4 shows that the major estrogens present in pregnant mare's urine do not include E₃. Rather, they include a number of other equine estrogens. Some of these equine estrogens are basically not produced in humans. Several of the equine estrogens contained in Premarin® are functionally similar to human pregnancy estrogens with respect to their preferential affinity for human ERβ over ERα.

Similarly, D-equilenin had a weaker binding affinity for human ERα than E₁ (RBA 20% of E₁), but its binding affinity for ERβ was much higher than that of E₁ (RBA 355% of E₁). Also, 17β-dihydroequilenin only had a weak binding affinity for ERα (RBA 10% of E₂), it retained very high binding affinity for ERβ (RBA 100% of E₂).

For example, Example 1 showed that equilin (i.e., 7-dehyro-E₁) had slightly decreased binding affinity for ERα compared to E₁ (its RBA 40% of E₁), but it had drastically increased binding affinity for ERβ (its RBA 631% of E₁). Similarly, D-equilenin had a much weaker binding affinity than E₁ for human ERα (RBA 20% of E₁), but its binding affinity for ERβ was more than 3 times higher than that of E₁. Also, while 17β-dihydroequilenin had a low binding affinity for ERα (35% of E₂), it had a high binding affinity for ERβ (RBA 100% of E₂). The binding affinities of 17β-dihydroequilin (i.e., 7-dehydro-E₂) for human ERα and ERβ were actually slightly higher than E₂ (its RBAs 142% and 113%, respectively, of E₂).

Further, while 6-dehydro-E₂ and 9(11)-dehydro-E₂ had slightly reduced binding affinity for ERα (RBAs 50% and 64% of E₂, respectively), their binding affinity for human ERβ was compared to E₂ (RBAs 89% and 119%, respectively, of E₂). Compared to E₁, 6-dehydro-E₁ had nearly the same binding affinity for ERβ, but its binding affinity for ERα was significantly decreased, with its RBA only 10% of E₁. 7-Dehyro-E₁ and 9(11)-dehydro-E₁ each had slightly decreased binding affinity for ERα compared to E₁ (RBAs 45% and 50% of E₁, respectively), but they had a drastically increased binding affinity for ERβ (RBAs 631% and 316% of E₁, respectively). Similarly, D-equilenin had a weaker binding affinity for human ERα than E₁ (RBA 20% of E₁), but its binding affinity for ERβ was much higher than that of E₁ (RBA 355% of E₁). Also, 17β-dihydroequilenin only had a weak binding affinity for ERα (RBA 35% of E₂), it retained very high binding affinity for ERβ (RBA 100% of E₂).

Taken together, it is evident that many of the equine estrogens contained in Premarin® have a preferential binding affinity for human ERβ over ERα.

### Example 5

### Hormone Replacement Formulations

In the present invention, a primary criterion that determines whether a given estrogen or combination of estrogens is ideal for postmenopausal hormone replacement therapy is that the estrogen(s) should be able to restore the hormonal environment to those in a normal non-pregnant young woman, but not that in a pregnant woman. Because very different types of estrogens are produced in pregnant compared to non-pregnant women and they serve very different physiological functions, it is theorized the use of endogenous estrogens found in a non-pregnant young woman would be more ideal for hormone replacement therapy than those predominantly produced during pregnancy.

In a preferred aspect, hormone replacement therapy formulation consisting essentially of estrogenic compounds such that: (1) the relative binding affinity for ERα (“RBA_{α}”) of the estrogenic compounds compared to 17β-estradiol (E₂) is less than about 100%; (2) the relative binding affinity for ERβ (“RBA_{β}”) of the estrogenic compounds compared to 17β-estradiol (E₂) is less than about 100%; and/or (3) the estrogenic compounds preferentially stimulate the ERα over the ERβ such that the ratio of RBA_{α}/RBA_{β} is greater than about 1. Most preferred estrogenic compounds are estrone (E₁), 17α-estradiol (17α-E₂), 2-hydroxyestrone (2-OH-E₁), 2-methoxyestrone (2-MeO-E₁), and/or 2-methoxyestradiol (2-MeO-E₂), as well as their sulfated or glucuronidated conjugates.

For example, one preferred hormone replacement formulation comprises about 0.1-0.3 mg estrone (E₁) sulfate, and/or about 0.1-0.3 mg 17α-estradiol (17α-E₂) sulfate, and/or about 0.1 to 0.5 mg 2-hydroxyestrone (2-OH-E₁) sulfate, and/or about 0.1 to 1 mg 2-methoxyestrone (2-MeO-E₁), and/or about 0.1 to 1 mg 2-methoxyestradiol (2-MeO-E₂).

It should also be noted that some endogenous estrogens (such as the conjugates of 2-methoxyestradiol) are beneficial antitumorigenic estrogen metabolites. Given that many of the endogenous estrogens may have a rather rapid metabolic disposition in the body, some other naturally-occurring or synthetic estrogens that have longer half-lives and can also provide a similar preferential activation of the ERα system as E₁ may also be useful as alternatives. For instance, since 17α-E₂ has similar ER-binding preference as E₁ but it cannot be readily converted to E₂ by 17β-hydroxysterpoid dehydrogenase, its sulfate conjugates may serve as alternatives to E₁ sulfate to achieve similar biological functions.

In the present invention, using conjugated estrogens, such as sulfated estrogens for human hormone replacement therapy is also preferable to using the corresponding parent estrogens. The main reasons are: (i) The sulfated estrogens are inactive themselves (with little or no binding affinity for human ERα and ERβ), but they can be enzymatically hydrolyzed to release bioactive estrogens in a variety of tissues in the body. As such, oral administration of estrogen sulfates would have the natural cushion effect which would avoid causing unwanted over-stimulation of the ER system throughout the body. Instead, they usually would only activate those target tissues or cells that are most in need of estrogenic stimulation. Here it is also of note that several recent studies have already shown that the estrogen target cells can actively transport E₁-3-sulfate into the cells. Moreover, these cells may selectively adjust their ability to actively transport E₁-3-sulfate into the cells to release bioactive estrogens, depending on the level of their hormonal needs. See Pizzagalli et al., Identification of Steroid Sulfate Transport Process in the Human Mammary Gland, J. Clin. Endocrinol. Metab. 88, 3902-3912 (2003). Theoretically, such a mechanism would offer certain degrees of target organ selectivity of estrogenic stimulation. Compared to estrogen glucuronides, estrogen sulfates are probably better because they usually have longer half-lives (t_{1/2}) in the body, thereby making them pharmacologically more useful.

Based on the discussion given above, it is suggested that modest levels of stimulation of both ERα and ERβ systems with a slight preference for the ERα system would be better for postmenopausal hormone replacement therapy than estrogens that confer a predominant activation of the ERβ system. It is apparent that Premarin®, the most widely prescribed hormone replacement therapy, is less ideal for achieving this clinical purpose. While there is considerable amount of E₁-3-sulfate contained in Premarin®, which presumably is good for its intended purpose as a hormone replacement therapy, the fact is that it also contains many other very potent pregnancy equine estrogens which would jointly produce a strong over-stimulation of the ERβ system. Similarly, genistein, a potent and preferential partial agonist of human ERβ, would be even less suitable than Premarin® for use in postmenopausal hormone replacement therapy because it would essentially provide a near selective ERβ stimulation. This suggestion is in agreement with recent clinical observations showing that the singular use of genistein is ineffective as a hormone replacement therapy in postmenopausal woman.

The following references to the extent that they provide exemplary procedural or other details supplementary to those set forth herein, are specifically incorporated herein by reference.
Anstead et al., 2-Arylindenes and 2-arylindenones: Molecular structures and considerations in the binding orientation of unsymmetrical nonsteroidal ligands to the estrogen receptor, J. Med. Chem. 32, 2163-2172 (1989).
Anstead et al., The estradiol pharmacophore: ligand structure-estrogen receptor binding affinity relationships and a model for the receptor binding site, Steroids 62, 268-303 (1997).
Ball et al., Catecholoestrogens (2- and 4-hydroxyoestrogens). Chemistry, biogenesis, metabolism, occurrence and physiological significance, Acta. Endocrinol. 232, 1-127 (1980).
Bradlow et al., Oestradiol 16α-hydroxylase: A risk marker for breast cancer, Cancer Surv. 5, 573-583 (1986).
Brooks et al., Estrogen structure-receptor function relationships, Moudgil V K (editor), Recent Advances in Steroid Hormone Action. Walter de Gruyter, Berlin 443-466 (1987).
Brzozowski et al., Molecular basis of agonism and antagonism in the oestrogen receptor, Nature 389, 753-758 (1997).
Cramer et al., Crossvalidation, bootstrapping, and partial least squares compared with multiple regression in conventional QSAR studies, Quant. Struct. Act. Relat. 7, 18-25 (1988).
Cushman et al., Synthesis, antitubulin and antimitotic activity, and cytotoxicity of analogs of 2-methoxyestradiol, an endogenous mammalian metabolite of estradiol that inhibits tubulin polymerization by binding to the colchicine binding site, J. Med. Chem. 38, 2041-2049 (1995).
Denger et al., Tissue-specific expression of human ERα and ERβ in the male, Mol. Cell. Endocrinol. 178, 155-160 (2001).
Enmark et al., Human estrogen receptor β-gene structure, chromosomal localization, and expression pattern, J. Clin. Endocrinol. Metab. 82, 4258-4265 (1997).
Fanchenko et al., The specificity of human estrogen receptor, Acta. Endocrinol. 7, 232-240 (1979).
Fang et al., Structure-activity relationships for a large diverse set of natural, synthetic, and environmental estrogens, Chem. Res. Toxicol. 14, 280-294 (2001).
Fishman et al., Biological properties of 16α-hydroxyestrone. Implications in estrogen physiology and pathophysiology, J. Clin. Endocrinol. Metab. 51, 611-615 (1980).
Fishman, Biological action of catecholestrogens. J. Endocr. 85, 59-65 (1981).
Fitzpatrick, Soy isoflavones. Hope or hype, Maturitas 44 (Suppl 1), S21-S29 (2003).
Gabbard et al., Structure-activity relationships of estrogens. Effects of 14-dehydrogenation and axial methyl groups at C-7, C-9, and C-11, Steroids 41, 791-804 (1983).
Gao et al., Comparative QSAR analysis of estrogen receptor ligands, Chem. Rev. 99, 723-744 (1999).
Green et al., Human oestrogen receptor cDNA: sequence, expression and homology to v-erb-A, Nature 320, 134-139 (1986).
Greene et al., Sequence and expression of human estrogen receptor complementary DNA, Science 231, 1150-1154 (1986).
Gruber et al., Production and actions of estrogens, N. Engl. J. Med. 346, 340-352 (2002).
Halgren, Merck molecular force field. I. Basis, form, scope, parameterization, and performance of MMFF94, J. Comput. Chem. 17, 490-519 (1996).
Hewitt et al., Oestrogen receptor knockout mice. Roles for oestrogen receptors α and β in reproductive tissues, Reproduction 125, 143-149 (2003).
Hewitt et al., Lessons in estrogen biology from knockout and transgenic animals, Annu. Rev. Physiol. 67, 285-308 (2005).
Jiang et al., Synthesis of 7α-substituted derivatives of 17β-estradiol. Steroids [in press] (2006).
Jordan, Tamoxifen: A most unlikely pioneering medicine, Nat. Rev. Drug Discov. 2, 205-213 (2003).
Kaspar et al., Shielding effects at 17α-substituted estrogens. A tentative explanation for the low biological activity of 17α-ethyl-estradiol based on IR and NMR spectroscopic studies, J. Steroid. Biochem. 23, 611-616 (1985).
Katzenellenbogen, Estrogen receptors: Bioactivities and interactions with cell signaling pathways, Biol. Reprod. 54, 287-293 (1996).
Katzenellenbogen et al., Photoaffinity labels for estrogen binding proteins of rat uterus, Biochemistry 12, 4085-4092 (1973).
Katzenellenbogen et al., In vivo and in vitro steroid receptor assays in the design of estrogen pharmaceuticals, In: Eckelman W C (editor), Receptor-Binding Radiotracers. Vol. 1. CRC, Boca Raton, Fla. 93-126 (1982).
Katzenellenbogen et al., Hormone binding and transcription activation by estrogen receptors: analyses using mammalian and yeast systems, J. Steroid. Biochem. Mol., Biol. 47, 39-48 (1993).
Katzenellenbogen et al., A new actor in the estrogen receptor drama—Enter ER-β, Endocrinology 138, 861-862 (1997).
Korenman, Comparative binding affinity, of estrogens and its relation to estrogenic potency, Steroids 13, 163-177 (1969).
Kuiper et al., Cloning of a novel receptor expressed in rat prostate and ovary, Proc. Natl. Acad. Sci. 93, 5925-5930 (1996).
Kuiper et al., Comparison of the ligand binding specificity and transcript tissue distribution of estrogen receptors α and β, Endocrinology 138, 863-870 (1997).
Labrie et al., The key role of 17β-hydroxysteroid dehydrogenases in sex steroid biology, Steroids 62, 148-158 (1997).
Lee et al., Characterization of the oxidative metabolites of 17β-estradiol and estrone formed by 15 selectively expressed human cytochrome p450 isoforms. Endocrinology 144, 3382-3398 (2003).
Lemmen et al., Expression of estrogen receptor α and β during mouse embryogenesis, Mech. Dev. 81, 163-167 (1999).
Liu et al., Concentration-dependent mitogenic and antiproliferative actions of 2-methoxyestradiol in estrogen receptor-positive human breast cancer cells, J. Steroid. Biochem, Mol. Biol. 88, 265-275 (2004).
MacLusky et al., Catechol estrogens and estrogen receptors, Catechol. Estrogens, Merriam G R and Lipsett M B (editors), 151-165, New York, Raven Press (I 983).
Manas et al., Structure-based design of estrogen receptor-β selective ligands, J. Am. Chem. Soc. 126, 15106-15119 (2004).
Martucci et al., P450 enzymes of estrogen metabolism, Pharmacol. Ther. 57, 237-257 (1993).
Mosselman et al., ERβ: Identification and characterization of a novel human estrogen receptor, 392, 49-53 (1996).
Nilsson et al., Mechanisms of estrogen action, Physiol. Rev. 81, 1535-1565 (2001).
Nilsson et al., Biological role of estrogen and estrogen receptors, Crit. Rev. Biochem. Mol. Biol. 37, 1-28 (2002).
Peltoketo et al., Regulation of estrogen action. Role of 17β-hydroxysteroid dehydrogenases, Vitam. Horm. 55, 35-98 (1999).
Reid et al., Human estrogen receptor-α: Regulation by synthesis, modification and degradation. CMLS Cell. Mol. Life. Sci. 59, 821-831 (2002).
Saunders, Effects of sex steroids and related compounds on pregnancy and on development of the young, Physiol. Rev. 48, 601-643 (1968).
Saunders et al., Expression of oestrogen receptor β (ERβ) in multiple rat tissues visualised by immunohistochemistry, J. Endocrinol. 154, R13-R16 (1997).
Shughrue et al., Comparative distribution of estrogen receptor-α and -βmRNA in the rat central nervous system, J. Comp. Neurol. 388, 507-525 (1997).
Spong et al., Maternal estrogen receptor-β expression during mouse gestation, Am. J. Reprod. Immunol. 44, 249-252 (2000).
Swaneck et al., Covalent binding of the endogenous estrogen 16α-hydroxyestrone to estradiol receptor in human breast cancer cells: Characterization and intranuclear localization, Proc. Natl. Acad. Sci. USA 85, 7831-7835 (1988).
Tsvetan, Quantitative structure-activity relationships/comparative molecular field analysis (QSAR/CoMFA) for receptor-binding properties of halogenated estradiol derivatives, J. Med. Chem. 37, 4164-4176 (1994).
Wakeling et al., ICI 182,780, a new antioestrogen with clinical potential, J. Steroid. Biochem. Mol. Biol. 43, 173-177 (1992).
Wang et al., Synthesis of B-ring homologated estradiol analogues that modulate tubulin polymerization and microtubule stability, J. Med. Chem. 43, 2419-2429 (2000).
Wold et al., Multivariate data analysis in chemistry, In Chemometrics: Mathematics and Statistics in Chemistry; Kowalski B, Ed, Reidel: Dordrecht, The Netherlands (1984).
Wolohan et al., CoMFA and docking study of novel estrogen receptor subtype selective ligands, J. Comput. Aided Mol. Des. 17, 313-328 (2003).
Zhu et al., Functional role of estrogen metabolism in target cells: Review and perspectives, Carcinogenesis 19, 1-27 (1998).
Zhu et al., Is 2-methoxyestradiol an endogenous estrogen metabolite that inhibits mammary carcinogenesis, Cancer Res. 58, 2269-2277 (1998).
Zhu, et al., NADPH-dependent metabolism of 17β-estradiol and estrone to polar and nonpolar metabolites by human tissues and cytochrome P450 isoforms, Steroids 70, 225-244 (2005).

From the foregoing, it will be seen that this invention is one well adapted to attain all ends and objectives herein above set forth, together with the other advantages which are obvious and which are inherent to the invention. Since many possible embodiments may be made of the invention without departing from the scope thereof, it is to be understood that all matters herein set forth herein are to be interpreted as illustrative, and not in a limiting sense. While specific embodiments have been shown and discussed, various modifications may of course be made, and the invention is not limited to the specific forms or arrangement of parts and steps described herein, except insofar as such limitations are included in the following claims. Further, it will be understood that certain features and subcombinations are of utility and may be employed without reference to other features and subcombinations. This is contemplated by and is within the scope of the claims.

What is claimed and desired to be secured by Letters Patent is as follows:

1. An estrogen formulation for use in hormone replacement therapy consisting essentially of:
a therapeutically effective amount of at least one or more estrogenic compounds which preferentially stimulate the estrogen receptor alpha (ERα) compared to the estrogen receptor beta (“ERβ”) and a pharmaceutically acceptable carrier.

2. The estrogen formulation of claim 1 consisting essentially of at least two estrogenic compounds which preferentially stimulate the estrogen receptor alpha (ERα) compared to the estrogen receptor beta (“ERβ”) and a pharmaceutically acceptable carrier.

3. The estrogen formulation of claim 1 consisting essentially of at least three estrogenic compounds which preferentially stimulate the estrogen receptor alpha (ERα) compared to the estrogen receptor beta (“ERβ”) and a pharmaceutically acceptable carrier.

4. The estrogen formulation of claim 3 wherein said estrogenic compounds have a relative binding affinity for ERα (“RBA_{α}”) compared to 17β-estradiol (E₂) which is less than about 100%.

5. The estrogen formulation of claim 3 wherein said estrogenic compounds have an RBA, compared to 17β-estradiol of less than about 30%.

6. The estrogen formulation of claim 5 wherein at least two of said at least three estrogenic compounds wherein said estrogenic compounds have an RBA_{α} compared to 17β-estradiol of about 10% or less.

7. The estrogen formulation of claim 3 wherein said estrogenic compounds have a relative binding affinity for ERβ (“RBA_{β}”) compared to 17β-estradiol (E₂) which is less than about 100%.

8. The estrogen formulation of claim 3 wherein said estrogenic compounds have an RBA_{β} compared to 17β-estradiol of less than about 10%.

9. The estrogen formulation of claim 8 wherein at least two of said at least three estrogenic compounds wherein said estrogenic compounds have an RBA_{β} compared to 17β-estradiol of about 5% or less.

10. The estrogen formulation of claim 3 wherein said estrogenic compounds have an RBA_{β} compared to 17β-estradiol of less than about 5%.

11. The estrogen formulation of claim 3 wherein said estrogenic compounds have a ratio of RBA_{α}/RBA_{β} which is greater than about 2.

12. The estrogen formulation of claim 3 wherein at least one of said estrogenic compounds has a ratio of RBA_{α}/RBA_{β} which is greater than about 5.

13. The estrogen formulation of claim 3 wherein at least one of said estrogenic compounds has a ratio of RBA_{α}/RBA_{β} which is greater than about 10.

14. The estrogen formulation of claim 1 wherein said estrogenic compounds are selected from the group consisting of estrone (RBA_{α}/RBA_{β} about 5), 1-methylestradiol (RBA_{α}/RBA_{β} about 1.8), 2-aminoestrone (RBA_{α}/RBA_{β} about 7.5), 2-nitroestrone (RBA_{α}/RBA_{β} about 3), 2-hydroxyestrone (RBA_{α}/RBA_{β} about 10), 2-methoxyestradiol (RBA_{α}/RBA_{β} about 2), 2-bromoestradiol (RBA_{α}/RBA_{β} about 10), 4-nitroestrone (RBA_{α}/RBA_{β} about 10); 4-hydroxyestrone (RBA_{α}/RBA_{β} about 2), 4-hydroxyestradiol (RBA_{α}/RBA_{β} about 1.3), 4-methoxyestradiol (RBA_{α}/RBA_{β} about 2), 6-ketoestrone ((RBA_{α}/RBA_{β} about 2), 6α-hydroxyestradiol (RBA_{α}/RBA_{β} about 1.5), 6-ketoestradiol (RBA_{α}/RBA_{β} about 1.3), 6-ketoestriol (RBA_{α}/RBA_{β} about 8.3), 6-ketoestradiol-17α (RBA_{α}/RBA_{β} about 2), 7-dehydroestradiol (RBA_{α}/RBA_{β} about 1.3), 7-dehydroestradiol-17α (RBA_{α}/RBA_{β} about 1.3), 2-hydroxyestriol (RBA_{α}/RBA_{β} about 2), 17β-estradiol 11-acetate (RBA_{α}/RBA_{β} of 1.2), 11-β-methoxyethynyl estradiol (RBA_{α}/RBA_{β} of about 1.8), estetrol (RBA_{α}/RBA_{β} of about 1.3), and 16β-hydroxyestradiol (RBA_{α}/RBA_{β} of about 1.3), 17α-estradiol (RBA_{α}/RBA_{β} about 7.3), 17α-ethynylestradiol (RBA_{α}/RBA_{β} about 3.6).

15. The estrogen formulation of claim 1 wherein said estrogenic compounds are selected from the group consisting of estrone (E₁), 17α-estradiol (17α-E₂), 2-hydroxyestrone (2-OH-E₁), 2-methoxyestrone (2-MeO-E₁), and 2-methoxyestradiol (2-MeO-E₂) and their corresponding conjugates, and pharmaceutically acceptable salts thereof.

16. The estrogen formulation of claim 3 wherein said estrogenic compounds are selected from the group consisting of estrone (E₁), 17α-estradiol (17α-E₂), 2-hydroxyestrone (2-OH-E₁), 2-methoxyestrone (2-MeO-E₁), and 2-methoxyestradiol (2-MeO-E₂) and their corresponding conjugates, and pharmaceutically acceptable salts thereof.

17. The estrogen formulation of claim 15 wherein said conjugates are sulfated or glucuronidated conjugates.

18. The estrogen formulation of claim 1 wherein said estrogenic compounds are endogenous to non-pregnant pre-menopausal human females.

19. The estrogen formulation of claim 1 wherein said formulation is in tablet form.

20. The estrogen formulation of claim 1 wherein said formulation consists of a therapeutically effective amount of three to five estrogenic compounds which preferentially stimulate the estrogen receptor alpha (ERα) compared to the estrogen receptor beta (“ERβ”) and a pharmaceutically acceptable carrier.

21. The estrogen formulation of claim 20 wherein said three to five estrogenic compounds are selected from the group consisting of estrone (E₁), 17α-estradiol (17α-E₂), 2-hydroxyestrone (2-OH-E₁), 2-methoxyestrone (2-MeO-E₁), and 2-methoxyestradiol (2-MeO-E₂) and their corresponding conjugates, and pharmaceutically acceptable salts thereof.

22. A method for the treatment of peri-menopausal or post-menopausal symptoms in a human female which comprises administering the estrogen formulation of claim 1.

23. The method of claim 22 wherein said formulation comprises at least three estrogenic compounds, said estrogenic compounds selected from the group consisting of estrone (E₁), 17α-estradiol (17α-E₂), 2-hydroxyestrone (2-OH-E₁), 2-methoxyestrone (2-MeO-E₁), and 2-methoxyestradiol (2-MeO-E₂) and their corresponding conjugates, and pharmaceutically acceptable salts thereof.

24. The method of claim 23 wherein said at least three estrogenic compounds are co-administered at the same time in a single formulation.
